(19) United States
(12) Patent Application Publication
(10) Pub. No.: US 2026/0132160 A1
(43) Pub. Date: May 14, 2026

(54) IRON REPLACEMENT TREATMENTS

(71) Applicant: The General Hospital Corporation, Boston, MA (US)

(72) Inventors: Eric M. Gale, Hingham, MA (US); Artur Sargun, Brighton, MA (US); Jodie Babitt, Newton, MA (US); Allison L. Fisher, Boston, MA (US)

(21) Appl. No.: 19/121,785

(22) PCT Filed: Oct. 16, 2023
(86) PCT No.: PCT/US2023/076998

§ 371 Date: Apr. 16, 2025

Related U.S. Application Data
(60) Provisional application No. 63/416,889, filed on Oct. 17, 2022.
(60) Provisional application No. 63/449,748, filed on Mar. 3, 2023.

(51) Int. Cl. C07F 15/02; A61K 31/555; C07C 229/14; C07C 229/24; C07C 229/36; C07C 229/38; C07D 213/65; C07D 213/89; C07F 9/38; C07F 9/58; C07F 9/6558

(52) U.S. Cl. CPC C07F 15/025; A61K 31/555; C07C 229/14; C07C 229/24; C07C 229/36; C07C 229/38; C07D 213/65; C07D 213/89; C07F 9/3817; C07F 9/58; C07F 9/65583

(57) ABSTRACT

The present application provides compounds and methods for iron replacement therapy.

### CLAIM OF PRIORITY

This application claims the benefit of U.S. Patent Application Ser. No. 63/416,889 filed on Oct. 17, 2022; and 63/449,748, filed on Mar. 3, 2023. The entire contents of the foregoing are hereby incorporated by reference.

### FEDERALLY SPONSORED RESEARCH OR DEVELOPMENT

This invention was made with Government support under Grant Nos. DK120663 and DK130004 awarded by the National Institutes of Health. The Government has certain rights in the invention.

### TECHNICAL FIELD

This disclosure relates to compounds and methods for iron replacement therapy. In particular, the disclosure relates to iron coordination complexes capable to directly transfer iron to the iron carrier protein transferrin upon administration to a patient suffering from any form of anemia or iron deficiency.

### BACKGROUND

Anemia is a common condition affecting around 40% of children and 30% of reproductive age women worldwide and can have major health consequences. Anemia is often multifactorial. For example, nutritional deficiencies, infection, chronic immune activation, kidney disease, and genetic mutations can all cause or contribute to anemia and chronic diseases are generally the most common etiologies of anemia. The consequences of anemia may range from immune system dysfunction, disturbances in the gastrointestinal tract, impaired thermoregulation and neurocognitive function. Moreover, untreated anemia can be a risk or a prognostic factor for other diseases, such as tuberculosis and heart failure. In the United States alone, the socioeconomic burden attributable to anemia differs according to the type and severity of the pre-existing comorbidities and is significant, exceeding $30,000/year for each affected individual.

### SUMMARY

The present disclosure is based, at least in part, on a realization that iron coordination complexes, such as those within the instant claims, are capable of directly transferring Fe³⁺ to the iron carrier protein transferrin. As such, in one example, administration of the Fe³⁺ coordination complex leads to an increase in percentage transferrin saturation, while simultaneously avoiding the release of toxic free iron ions in the patient's blood stream that are not bound to transferrin or any other plasma protein (also referred to as “labile iron”), which typically results from direct iron supplementation (such as intravenous infusion of ferrous iron salts). The compounds are therefore advantageously useful for effective, efficient, and safe “direct to transferrin” iron replacement therapy. The compounds and methods within the instant claims allow for parenteral iron supplementation in patients suffering from iron deficiency anemia, including anemia related to inflammatory disease states where endogenous pathways of iron mobilization are severely inhibited, rendering iron replacement therapy challenging. This supplementation advantageously leads to alleviation of symptoms associated with anemia (such as fatigue, pale skin, shortness of breath, lightheadedness, dizziness, or a fast heartbeat) and complete recovery without any substantial adverse events or any substantial risk or iron poisoning.

In one general aspect, the present disclosure provides a compound which is a Fe³⁺ complex of Formula (A-I):

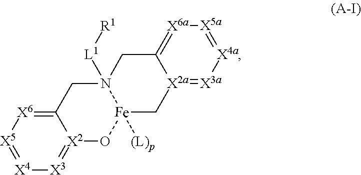

or a pharmaceutically acceptable salt thereof.

In another general aspect, the present disclosure provides a compound which is a Fe³⁺ complex of Formula (B-I):

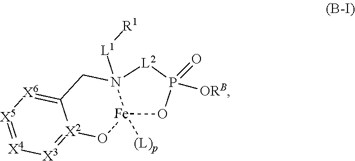

or a pharmaceutically acceptable salt thereof.

In yet another general aspect, the present disclosure provides a compound which is a Fe³⁺ complex of Formula (C-I):

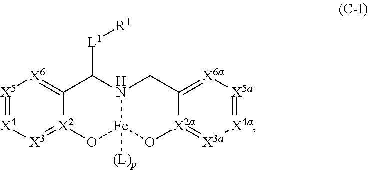

or a pharmaceutically acceptable salt thereof.

In yet another general aspect, the present disclosure provides a compound which is a Fe³⁺ complex of Formula (D-I):

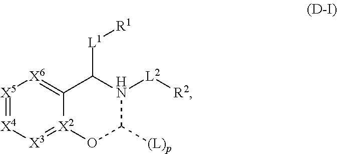

or a pharmaceutically acceptable salt thereof.

In yet another general aspect, the present disclosure provides a pharmaceutical composition comprising the iron coordination complex as described herein, or a pharmaceutically acceptable salt thereof, and a pharmaceutically acceptable carrier.

In yet another general aspect, the present disclosure provides a method of treating iron deficiency anemia, the method comprising administering to the subject in need thereof a therapeutically effective amount of the iron coordination complex as described herein, or a pharmaceutically acceptable salt thereof, or a pharmaceutical composition comprising same.

In yet another general aspect, the present disclosure provides a compound of Formula (A):

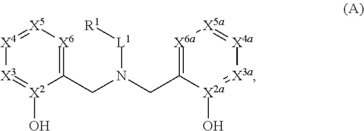

or a salt thereof.

In yet another general aspect, the present disclosure provides a compound of Formula (B):

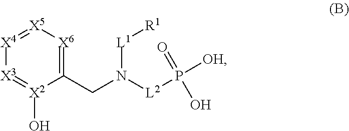

or a salt thereof.

In yet another general aspect, the present disclosure provides a compound of Formula (C):

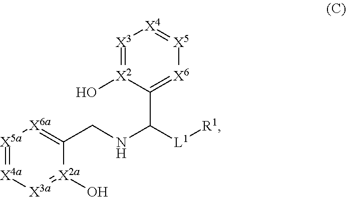

or a salt thereof.

In yet another general aspect, the present disclosure provides a compound of Formula (D):

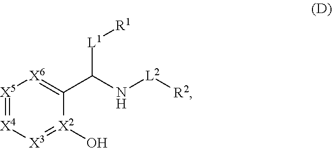

or a salt thereof.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1A shows a synthetic scheme showing preparation of compound BBG (See Example 1).

FIG. 1B shows a synthetic scheme showing preparation of compound Fe-BBG

### (See Example 2).

FIG. 2A shows HPLC traces of Fe-BBG with detection and 220 nm (top) and 454 nm (bottom), The HPLC method uses a Waters X-Bridge C18 column (3.5 μm; 4.6×150 mm) and the following method: The methods used on these systems are as follows: (A1) eluent A: 10 mM ammonium acetate in water, eluent B: 90:10 acetonitrile: MeCN: 10 mM ammonium acetate in water gradient: 5% B for 2 min, 5% B to 95% B over 10 min, 95% B for 1 min, 95% B to 5% B for 1 min, then 5% B for 1 min; flow rate 0.7 mL/min.

FIG. 2B shows the UV-vis spectrum of Fe-BBG. A photograph of a 0.1 mM solution of Fe-BBG is shown in the inset.

FIG. 3A-F shows solutions of 0.1 mM Fe-BBG incubated with between 0 and 100 molar equivalent bicarbonate (panel A), acetate (panel B), lactate (panel C), phosphate (panel E), and 0 to 10 molar equivalents citrate (panel F). The data indicate that BBG binds Fe³⁺ with substantially higher affinity than endogenous chelators often implicated as comprising labile Fe and non-transferrin bound iron.

FIG. 4A shows fluorescence spectra of 20 μM apo-transferrin after 2 h incubation with either 0, 0.5, 1, or 2 molar equivalents Fe-BBG (Conditions: 50 mM HEPES buffer supplemented with 25 mM bicarbonate, pH 7.4, 37° C.).

FIG. 4B shows rea gel electrophoresis separation of apo-, mono-ferric-, and diferric (holo)-transferrin from mixtures containing 10 μM apo-transferrin incubated with 0, 1, 2, 5, and 10 equiv, Fe-BBG. and confirms stoichiometric Fe³⁺ transfer of 2 molar equivalents Fe to apo-transferrin. Incubation with FPC was positive control, the stable compound Fe-PyC3A was negative control (Conditions: 50 mM HEPES buffer supplemented with 25 mM bicarbonate, pH 7.4, 37° C.).

FIG. 5 shows the time course of the Fe³⁺ trans-chelation reaction recorded via fluorescence emission intensity at 330 nm (λₑₓ=280 nm, instrument temporal resolution=6 s), and shows that Fe³⁺ transfer is complete within 6 s of mixing (Conditions: 50 mM HEPES buffer supplemented with 25 mM bicarbonate, pH 7.4, room temperature).

FIG. 6 shows UV-vis spectra of 0.1 mM Fe-BBG incubated without or with 1 molar equivalent ascorbate at pH 7.4 (pH 7.4, 50 mM HEPES buffer).

FIG. 7 shows comparison of thiobarbituric acid reactive substances generated by incubated Fe-BBG and other iron complexes (ferric pyrophosphate citrate, Fe-NTA. Fe-EDTA) under the following conditions: 5 mM Fe³⁺ complex, 2.8 mM H₂O₂, 2.8 mM deoxyribose, 0.1 mM ascorbic acid, 100 mM pH 7.4 phosphate buffer, 37° C.). The data demonstrate that Fe-BBG does not engage in redox cycling and generate Fenton radicals. The assay is performed as described in Methods in Enzymology 1994, 233, 57-66.

FIG. 8A-C shows coronal T1-weighted magnetic resonance images acquired after injection of 0.14 mmol/kg Fe-BBG into a mouse. Panel A shows liver enhancement and gallbladder enhancement (yellow arrow) consistent with partial hepatobiliary excretion. Panel B shows early and rapidly diminishing kidney enhancement, consistent with rapid elimination through the kidneys. Panel C plots the time course of signal intensity recorded in the vena cava, from monoexponential fits to this data a blood elimination half life of 5.5±2.8 min is estimated.

FIG. 9A-D shows serum Fe parameters recorded ex vivo in serum harvested from mice 5, 60, and 100 min after injection of either 0.56 μg Fe per g animal or 5.6 μg Fe per g animal as Fe-BBG. The data demonstrate that Fe-BBG injection rapidly raises serum Fe (panel A), and total iron binding capacity (panel B), does not reduce UIBC significantly, likely due to the presence of BBG ligand (panel C), and elevates % TSAT significantly (panel D) compared to placebo treated mice.

FIG. 10 shows quantitation of labile iron in serum serum harvested from mice 5, 60, and 100 min after injection of either 0.56 μg Fe per g animal or 5.6 μg Fe per g animal as Fe-BBG. These are the same serum samples as in FIG. 9. The assay was performed as described in Blood 2003, 102 (7), 2670-2677. The data demonstrates that Fe-BBG does not contribute detectable quantities of labile Fe, even 5 min after injection of the 5.6 μg Fe per g animal dose, when total serum Fe far exceeds TBIC.

FIG. 11A shows that solutions of 20 μM holo-transferrin and BBG of concentration ranging between 0 and 100 μM (0-5 molar equivalents) were incubated for 24 h in pH 7.4 50 mM HEPES buffer supplemented with 25 mM carbonate at 37° C., and Fe removal from transferrin was monitored using fluorescence spectroscopy (excitation wavelength: 280 nm). No evidence of Fe removal from transferrin was observed.

FIG. 11B shows that 10 μM holo-transferrin and was incubated with 100 μM BBG (10 molar equivalents) for 24 h in pH 7.4 50 mM HEPES buffer supplemented with 25 mM carbonate at 37° C., and Fe removal monitored by urea gel electrophoresis assay. No evidence of Fe removal from transferrin was observed.

FIG. 12 shows cyclic voltammograms of pH 7.4 50 mM HEPES buffer solution the absence (upper trace) and presence of 10 mM Fe-BBG (lower trace). Glassy carbon working electrode, Pt counter electrode, electrolyte: 0.5M KNO₃, scan rate=300 mV/s, RT.

FIG. 13A shows the aqueous solutions thermodynamics of BBG and its corresponding complexes of Fe³⁺, Cu²⁺, and Zn²⁺ that were interrogated through pH-potentiometric titration of BBG (L) in the absence and presence of 1 molar equivalent of Fe³⁺, Cu²⁺, or Zn²⁺.

FIG. 13B shows the entirety of the thermodynamics parameters tabulated. The conditional pH 7.4 stability constant of Fe-BBG (log K_{FeL PH 7.4}) is 19.51, whearas fo Cu-BBG and Zn-BBG, the log K_{CuL PH 7.4} and log K_{ZnL PH 7.4} values are 10.49 and 4.49, respectively. BBG binds Fe³⁺ with 9-orders of magnitude greater stability than Cu²⁺, and 15 orders of magnitude greater stability than Zn²⁺.

FIG. 14 shows human blood plasma samples containing between 0-5 mM BBG were incubated for 1 h at 37° C. before separation of the low-molecular weight solution components by ultrafiltration through a 10 kDa molecular weight cutoff filter. The plasma concentrate and ultrafiltrate were assayed for concentrations of Cu and Zn, as well as Mn, Mg, and Ca by ICP-MS.

FIG. 15 contains a table showing results of bone marrow and liver tissue analysis by qPCR for expression of genes related to erythropoietic activity, Fe exposure, oxidative stress, and inflammation. Fe-BBG did not trigger upregulation of liver heme oxygenase 1 (Hmox1), glutamate-cysteine ligase catalytic subunit (Gclc), or NAD (P) H quinone dehydrogenase 1 (NqoI), which taken together indicate that Fe-BBG did not result in oxidative stress. Expression of Serum amyloid A1 (SaaI), a sensitive marker of inflammation, was also unchanged after repeat dosing with Fe-BBG.

FIG. 16 shows that repeat dosing of Fe-BBG to Tmprss6 knockout mice did not generate significant differences in any clinical chemistry parameters, including serum markers routinely used in drug toxicity screens.

FIG. 17 shows a synthetic scheme showing preparation of compound SBBG (See Example 17).

FIG. 18 shows an HPLC trace of SBBG with detection at 280 nm (top). An MS chromatogram of detected m/z−=366, corresponding the SBBG anion, is shown on the bottom. The HPLC method uses a Waters X-Bridge C18 column (3.5 μm; 4.6×150 mm) and the following method: eluent A: 10 mM ammonium acetate in water, eluent B: 90:10 acetonitrile: MeCN: 10 mM ammonium acetate in water gradient: 5% B for 2 min, 5% B to 95% B over 10 min, 95% B for 1 min, 95% B to 5% B for 1 min, then 5% B for 1 min; flow rate 0.7 mL/min.

FIG. 19 shows a synthetic scheme showing preparation of the compound Fe-SBBG (see Example 18)

FIG. 20 shows HPLC traces of Fe-SBBG with detection and 254 nm (top) and 465 nm (middle). An MS chromatogram of detected m/z⁻=455, corresponding the [Fe(SBBG)(H₂O)₂]⁻ ion is shown on the bottom. The HPLC method uses a Phenomenex Luna C18 column (3.5 μm; 4.6×100 mm) and the following method: eluent A: 0.1% formic acid in water, eluent B: 90:10 acetonitrile: 0.1% formic acid in water: gradient: 5% B to 40% B over 4 min, 40% B to 95% B over 1 min, 95% B to 5% B for 1 min, then 5% B for 2 min; flow rate 1.0 mL/min. (See Example 18)

FIG. 21 shows a synthetic scheme showing preparation of the compound BBG-COOH (see Example 19)

FIG. 22 shows an HPLC trace of BBG-COOH with detection at 80 nm (top). An MS chromatogram of detected m/z⁻=330, corresponding the anionic form of BBG-COOH is shown on the bottom. The HPLC method uses a Waters X-Bridge C18 column (3.5 μm; 4.6×150 mm) and the following method: eluent A: 10 mM ammonium acetate in water, eluent B: 90:10 acetonitrile: MeCN: 10 mM ammonium acetate in water gradient: 5% B for 2 min, 5% B to 95% B over 10 min, 95% B for 1 min, 95% B to 5% B for 1 min, then 5% B for 1 min; flow rate 0.7 mL/min. (See Example 19).

FIG. 23 shows a synthetic scheme showing preparation of the compound Fe-BBG-COOH (see Example 20)

FIG. 24 shows HPLC traces of Fe-BBG-COOH with detection and 254 nm (top) and 465 nm (middle). An MS chromatogram of detected m/z⁻=419, corresponding the anion [Fe(BBG-COOH)(H₂O)₂]⁻ is shown on the bottom. The HPLC method uses a Phenomenex Luna C18 column (3.5 μm; 4.6×100 mm) and the following method: eluent A: 0.1% formic acid in water, eluent B: 90:10 acetonitrile: 0.1% formic acid in water: gradient: 5% B to 40% B over 4 min, 40% B to 95% B over 1 min, 95% B to 5% B for 1 min, then 5% B for 2 min; flow rate 1.0 mL/min. (See Example 20).

### DETAILED DESCRIPTION

Several forms of anemia, including anemia of inflammation (AI) and iron-refractory iron deficiency anemia (IRIDA) are caused in part by pathologic iron (Fe) restriction. In these conditions, chronic immune activation or genetic mutations upregulate the Fe regulating hormone hepcidin, which in turn inhibits activity of ferroportin, the only known Fe exporter. Hepcidin excess thus imposes a severe form of hypoferremia as Fe liberated via hemoglobin recycling in macrophages, nutritional iron absorbed by enterocytes, and other stored Fe cannot be exported to the plasma iron-carrier protein transferrin for distribution.

Fe replacement in patients with hepcidin excess can be challenging. Most intravenous Fe replacement drugs are Fe-carbohydrate nanoparticles that are accumulated and metabolized in macrophages, requiring ferroportin for Fe mobilization. Thus, I.V. iron replacement can simultaneously have limited efficacy for anemia correction while potentially contributing to iron overload in macrophages. Hepcidin-driven restriction also limits the efficacy of nutritional Fe supplements as Fe export from enterocytes is also ferroportin-mediated. Under hepcidin-inhibited conditions, administered Fe builds to toxic levels in enterocytes while anemia remains largely untreated.

There are no commercially available hepcidin-modulating drugs.

Another approach is to deliver Fe to transferrin via mechanisms independent of ferroportin. One method to directly deliver Fe to transferrin is via intravenous infusions of labile iron salts, but care must be taken to note overwhelm serum total iron binding capacity, otherwise exposure to toxic labile iron can occur. Typically, labile iron formulations are infused to patients over the course of hours in order to deliver therapeutic quantities of iron without overwhelming serum total iron binding capacity. The present disclosure advantageously provides compounds that are iron complexes that are useful as iron replacement drugs. These iron complexes are designed to stabilize iron in the trivalent oxidation state (Fe³⁺), to deliver Fe directly and stoichiometric ally to transferrin, and to be stable against trans-chelation with ligands implicated in in vivo speciation of labile iron and non-transferrin bound iron. Experimental data in this disclosure provides credible evidence that these complexes can be safely and efficiently administered to either dialysis or non-dialysis patients via fast, simple IV injection. Importantly here, the iron complexes within the instant claims can be administered safely at a very high initial dose so that the concentration of the iron complex in blood serum far exceeds serum transferrin concentration. Without being bound by any particular theory or speculation, it is believed that upon entering the bloodstream, the iron complex within the present claims selectively transfers iron to the protein transferrin, but otherwise retains the chelated iron ions to substantially avoid “labile iron” in the bloodstream, keeping the free iron ion concentration well beneath thresholds required for toxicity. If the iron complex is dosed so that serum concentrations are sufficiently high, the complex should continuously replenish transferrin-iron over the course of several transferrin-Fe half-lives. Some embodiments of the iron complexes, as well as the ligands that form these complexes, are described herein. Pharmaceutical formulations containing the iron complexes, and methods of using the iron complexes to treat, e.g., iron deficiency anemia, are described herein.

### Compounds of Formula (A)

In some embodiments, the present application provides a compound of Formula (A), which may be useful as a ligand for chelating iron ions (e.g., Fe³⁺) to form an iron complex as described herein. In some embodiments, the compound of Formula (A) has formula:

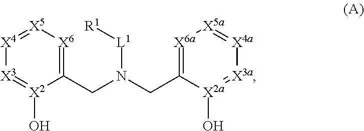

or a salt (e.g., a pharmaceutically acceptable salt) thereof, wherein:
L¹ is —C₁₋₃ alkylene-, optionally substituted with R¹⁰;
each R¹⁰ is independently —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, or —(C₁₋₃ alkyl)_{q}(C═O)NHSO₂R⁸;
R¹ is selected from OH, CO₂R^{A}, P(O)(OR^{A})(OR^{B}), and (C═O)NR^{A}R¹⁰;
each q is independently 0 or 1;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X³, and X⁶ are N;
X^{2a} is selected from N and C;
X^{3a} is selected from N and CR^{3a};
X^{4a} is selected from N and CR^{4a};
X^{5a} is selected from N and CR^{5a};
X^{6a} is selected from N and CR^{6a};
provided that no more than two of X^{2a}, X^{3a}, X^{4a}, X^{5a}, and X^{6a} are N;
R³, R^{3a}, R⁴, R^{4a}, R⁵, R^{5a}, R⁶, and R^{6a} are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹;

and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN,

In some embodiments, if L¹R¹ is CH₂C(═O)OR^{A} or CH(CH₃)C(═O)OR^{A}, then:

X² is different from X^{2a}, or X³ is different from X^{3a}; or X⁴ is different from X^{4a}, or X⁵ is different from X^{5a}, or X⁶ is different from X^{6a}; or X² is N; or X^{2a} is N. In some of the foregoing embodiments, L′R¹ is CH₂CH₂C(═O)OR^{A}.

In some embodiments, X² is different from X²a. In some embodiments, X³ is different from X^{3a}. In some embodiments, X⁴ is different from X^{4a}. In some embodiments, X⁵ is different from X⁵a. In some embodiments, X⁶ is different from X^{6a}.

In some embodiments, the compound of Formula (A) is not any one of the following compounds:

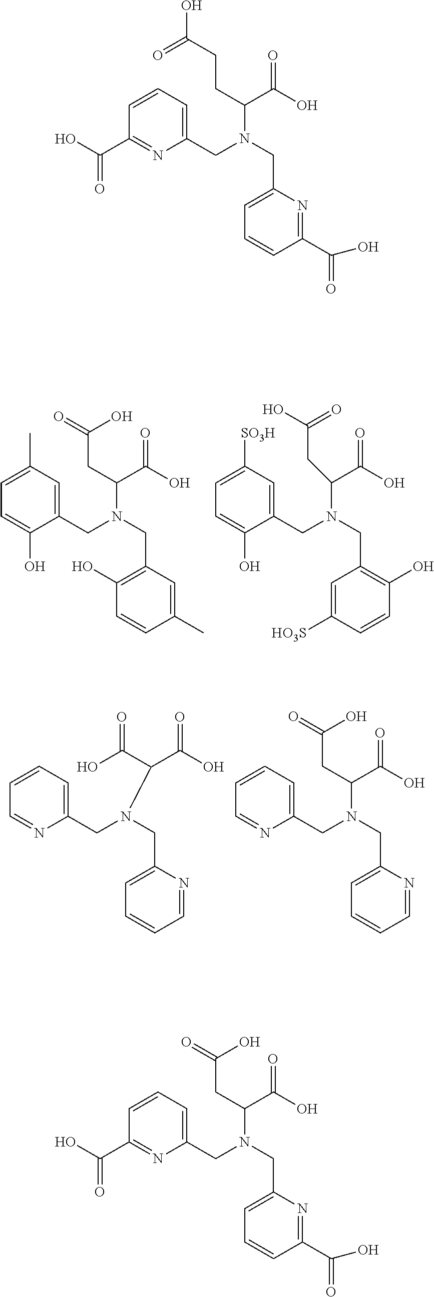

In some embodiments, X² is N. In some embodiments, X² is C. In some embodiments, X³ is N. In some embodiments, X³ is CR³. In some embodiments, X⁴ is N. In some embodiments, X⁴ is CR⁴. In some embodiments, X⁵ is N. In some embodiments, X⁵ is CR⁵. In some embodiments, X⁶ is N. In some embodiments, X⁶ is CR⁶.

In some embodiments, X^{2a} is N. In some embodiments, X^{2a} is C. In some embodiments, X^{3a} is N. In some embodiments, X^{3a} is CR³a. In some embodiments, X^{4a} is N. In some embodiments, X^{4a} is CR⁴a. In some embodiments, X^{5a} is N. In some embodiments, X^{5a} is CR⁵a. In some embodiments, X^{6a} is N. In some embodiments, X^{6a} is CR^{6a}.

In some embodiments, the compound of Formula (A) has formula:

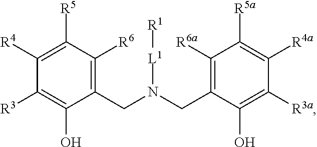

or a pharmaceutically acceptable salt thereof.

In some embodiments, the compound of Formula (A) has formula:

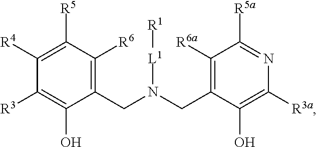

or a pharmaceutically acceptable salt thereof.

In some embodiments, the compound of Formula (A) has formula:

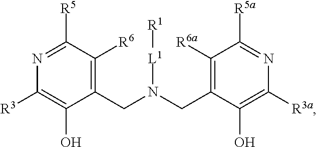

or a pharmaceutically acceptable salt thereof.

In some embodiments, L¹ is —C₁₋₃ alkylene-. In some embodiments, L¹ is —C₁₋₃ alkylene-substituted with R¹⁰. In some embodiments, L¹ is selected from methylene and ethylene. In some embodiments, L¹ is methylene. In some embodiments, L¹ is 1,2-ethylene. In some embodiments, L¹ is propylene.

In some embodiments, the compound has formula:

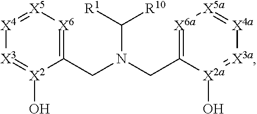

or a pharmaceutically acceptable salt thereof.

In some embodiments, the compound has formula:

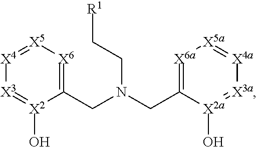

or a pharmaceutically acceptable salt thereof.

In some embodiments, the compound has formula:

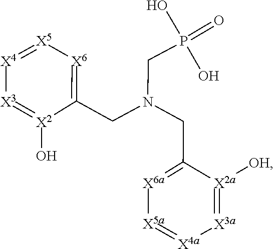

or a pharmaceutically acceptable salt thereof.

In some embodiments, R¹ is selected from CO₂R^{A}, P(O)(OR^{A})(OR^{B}), and (C═O)NR^{A}R¹⁰. In some embodiments, R¹ is OH. In some embodiments, R¹ is P(O)(OR^{A})(OR^{B}). In some embodiments, R¹ is P(O)(OH)₂. In some embodiments, R¹ is CO₂R^{A}. In some embodiments, R¹ is C(O)OH. In some embodiments, R¹ is (C═O)NR^{A}R¹⁰. In some embodiments, R¹ is (C═O)NR^{A}R¹⁰.

In some embodiments, R³, R⁴, R⁵, and R⁶ are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰. In some embodiments, R³, R⁴, R⁵, and R⁶ are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, and —(C₁₋₃ alkyl)_{q}CN. In some embodiments, at least one of R³, R⁴, R⁵, and R⁶ is selected from C₁₋₃ alkyl, C₁₋₃ alkoxy, C₁₋₃ haloalkyl, C₁₋₃ haloalkoxy, halo, —(C₁₋₃ alkyl)_{q}OH, and —(C₁₋₃ alkyl)_{q}CN. In some embodiments, at least one of R³, R⁴, R⁵, and R⁶ is R¹⁰.

In some embodiments, R^{3a}, R^{4a}, R^{5a}, and R^{6a} are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰. In some embodiments, R^{3a}, R^{4a}, R^{5a}, and R^{6a} are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, and —(C₁₋₃ alkyl)_{q}CN. In some embodiments, at least one of R^{3a}, R^{4a}, R^{5a}, and R^{6a} is selected from C₁₋₃ alkyl, C₁₋₃ alkoxy, C₁₋₃ haloalkyl, C₁₋₃ haloalkoxy, halo, —(C₁₋₃ alkyl)_{q}OH, and —(C₁₋₃ alkyl)_{q}CN. In some embodiments, at least one of R^{3a}, R^{4a}, R^{5a}, and R^{6a} is R¹⁰.

In some embodiments, at least one, at least two, or at least three of R³, R^{3a}, R⁴, R^{4a}, R⁵, R^{5a}, R⁶, and R^{6a} are each independently selected from —(C₁₋₃ alkyl)_{q}CO₂R^{A}, —(C₁₋₃ alkyl)_{q}OP(R⁸)O₂R^{B}, —(C₁₋₃ alkyl)_{q}(C═O)NHSO₂R⁸, and —(C₁₋₃ alkyl)_{q}PO₃R^{A}R⁸. In some embodiments, at least one, at least two, or at least three of R³, R^{3a}, R⁴, R^{4a}, R⁵, R^{5a}, R⁶, and R^{6a} are independently selected from C₁₋₃ alkyl-OPO(OR^{A})(OR^{B}), —C₁₋₃ alkyl-CO₂R^{A}, —C₁₋₃ alkyl-SO₃R^{A}, and —C₁₋₃ alkyl-(C═O)NR^{A}R^{B}. In some embodiments, at least one, at least two, or at least three of R³, R^{3a}, R¹, R^{4a}, R⁵, R^{5a}, R⁶, and R^{6a} are independently selected from C₁₋₃ alkyl-OPO(OH)(OH), —C₁₋₃ alkyl-C(O)(OH), —C₁₋₃ alkyl-SO₂ (OR^{A}), and —C₁₋₃ alkyl-(C═O)NH₂.

In some embodiments, R⁵ is selected from H, C₁₋₃ alkyl, C₁₋₃ alkoxy, C₁₋₃ haloalkyl, C₁₋₃ haloalkoxy, halo; and R^{5a} is selected from —(C₁₋₃ alkyl)_{q}OH, —(C₁₋₃ alkyl)_{q}CN, and R¹⁰.

In some embodiments, R³ is selected from H, C₁₋₃ alkyl, C₁₋₃ alkoxy, C₁₋₃ haloalkyl, C₁₋₃ haloalkoxy, halo; and R^{3a} is selected from —(C₁₋₃ alkyl)_{q}OH, —(C₁₋₃ alkyl)_{q}CN, and R¹⁰.

In some embodiments, R⁴ is selected from H, C₁₋₃ alkyl, C₁₋₃ alkoxy, C₁₋₃ haloalkyl, C₁₋₃ haloalkoxy, halo; and R^{4a} is selected from —(C₁₋₃ alkyl)_{q}OH, —(C₁₋₃ alkyl)_{q}CN, and R¹⁰.

In some embodiments, R⁶ is selected from H, C₁₋₃ alkyl, C₁₋₃ alkoxy, C₁₋₃ haloalkyl, C₁₋₃ haloalkoxy, halo; and R^{6a} is selected from —(C₁₋₃ alkyl)_{q}OH, —(C₁₋₃ alkyl)_{q}CN, and R¹⁰.

In some embodiments, R¹⁰ is selected from —(C₁₋₃ alkyl)_{q}CO₂R^{A}, —(C₁₋₃ alkyl)_{q}OP(R⁸)O₂R^{B}, —(C₁₋₃ alkyl)_{q}(C═O)NHSO₂R⁸, and —(C₁₋₃ alkyl)_{q}PO₃R^{A}R⁸.

In some embodiments, R¹⁰ is —(C₁₋₃ alkyl)_{q}CO₂R^{A}. In some embodiments, R¹⁰ is —(C₁₋₃ alkyl)_{q}CO₂H. In some embodiments, R¹⁰ is —(C₁₋₃ alkyl)_{q}OP(R⁸)O₂R^{B}. In some embodiments, R¹⁰ is —(C₁₋₃ alkyl)_{q}(C═O)NHSO₂R⁸. In some embodiments, R¹⁰ is —(C₁₋₃ alkyl)_{q}PO₃R^{A}R⁸.

In some embodiments, R¹⁰ is —(C₁₋₃ alkyl)_{q}SO₃R^{A}. In some embodiments, R¹⁰ is —(C₁₋₃ alkyl)_{q}SO₂R⁸. In some embodiments, R¹⁰ is —(C₁₋₃ alkyl)_{q}NHSO₂R⁸. In some embodiments, R¹⁰ is —(C₁₋₃ alkyl)_{q}NR^{A}R^{B}. In some embodiments, R¹⁰ is —(C₁₋₃ alkyl), (C═O)NR^{A}R^{B}. In some embodiments, R¹⁰ is —(C₁₋₃ alkyl)_{q}OPO₃R^{A}R^{B}.

In some embodiments, q is 0. In some embodiments, q is 1.

In some embodiments, R⁸ is C₃₋₉ alkyl. In some embodiments, R⁸ is —(C₁₋₃ alkyl)_{q}C₆₋₁₀ aryl, wherein said C₆₋₁₀ aryl is optionally substituted with 1, 2, or 3 substituents independently selected from R¹¹. In some embodiments, R¹¹ is selected from C₁₋₆ alkyl, C₁₋₆ alkoxy, C₁₋₃ haloalkyl, C₁₋₃ haloalkoxy, halo, OH, and CN. In some embodiments, R¹¹ is and C₆₋₁₀ aryl, optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN.

In some embodiments, R⁸ is selected from:

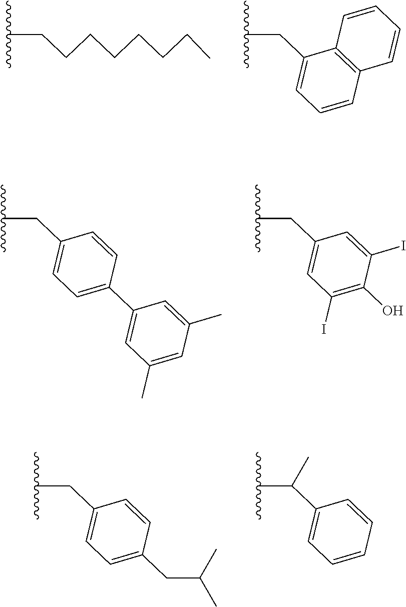

In some embodiments, the compound of Formula (A) is selected from any one of the following compounds:

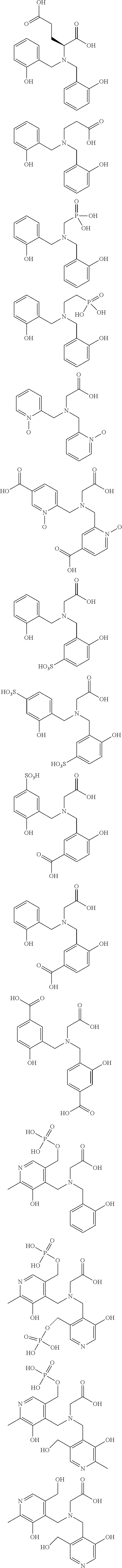

or a pharmaceutically acceptable salt thereof.

In some embodiments, the compound of Formula (A) is selected from any one of the following compounds:

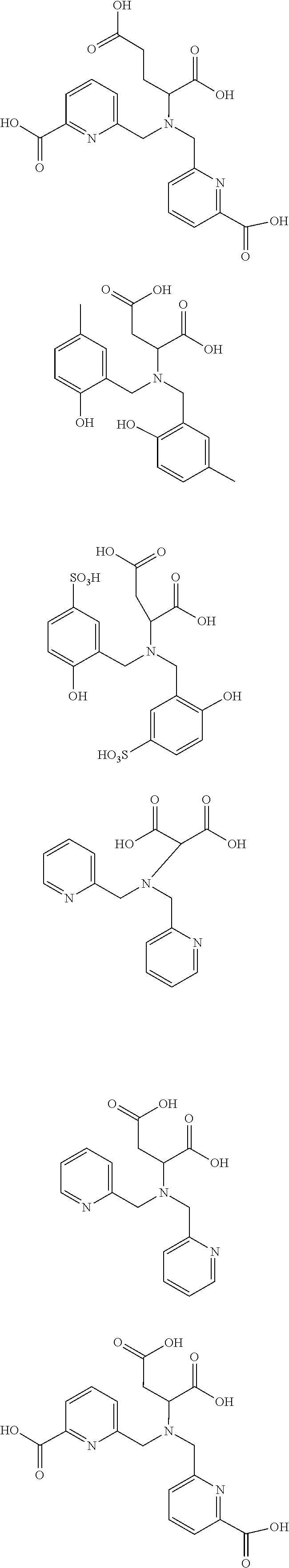 [SMILES: O=C(O)CCC(C(=O)O)N(Cc1cccc(C(=O)O)n1)Cc1cccc(C(=O)O)n1 ; Cc1ccc(O)c(CN(Cc2cc(C)ccc2O)C(CC(=O)O)C(=O)O)c1 ; O=C(O)CC(C(=O)O)N(Cc1cc(S(=O)(=O)O)ccc1O)Cc1cc(S(=O)(=O)O)ccc1O ; O=C(O)C(C(=O)O)N(Cc1ccccn1)Cc1ccccn1 ; O=C(O)CC(C(=O)O)N(Cc1ccccn1)Cc1ccccn1 ; O=C(O)CC(C(=O)O)N(Cc1cccc(C(=O)O)n1)Cc1cccc(C(=O)O)n1]

or a pharmaceutically acceptable salt thereof.

### Compounds of Formula (I)

In some embodiments, the compound of Formula (A) as described herein encompasses a compound of Formula (I). Hence, in some embodiments, the present disclosure provides a compound of Formula (I):

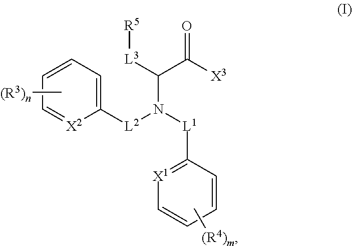

or a pharmaceutically acceptable salt thereof. In some embodiments, the Formula (I) comprises any combination of L¹, L², L³, X¹, X², X³, n, R³, R⁴, and R⁵ as described for any of the Formulae herein (including, but not limited to, Formula A and Formula I).

In some embodiments:
L¹ is C₁₋₃ alkylene;
L² is C₁₋₃ alkylene;
X¹ is selected from N and CR¹;
R¹ is selected from OH, SH, NH₂, C₁₋₃ alkylamino, and di(C₁₋₃ alkyl)amino;
n is 0, 1, 2, 3, or 4;
each R³ is independently selected from OH, NO₂, CN, halo, S(═O)₂OH, S(═O)₂NH₂, C₁₋₃ alkyl, C₁₋₃ haloalkyl, C₁₋₃ alkoxy, C₁₋₃ haloalkoxy, cyano-C₁₋₃ alkylene, HO—C₁₋₃ alkylene, amino, C₁₋₃ alkylamino, di(C₁₋₃ alkyl)amino, carboxy, and C₁₋₃ alkoxycarbonyl;
X² is selected from N and CR²;
R² is selected from OH, SH, NH₂, C₁₋₃ alkylamino, and di(C₁₋₃ alkyl)amino;
m is 0, 1, 2, 3, or 4;
each R⁴ is independently selected from OH, NO₂, CN, halo, S(═O)₂OH, S(═O)₂NH₂, C₁₋₃ alkyl, C₁₋₃ haloalkyl, C₁₋₃ alkoxy, C₁₋₃ haloalkoxy, cyano-C₁₋₃ alkylene, HO—C₁₋₃ alkylene, amino, C₁₋₃ alkylamino, di(C₁₋₃ alkyl)amino, carboxy, and C₁₋₃ alkoxycarbonyl;
X³ is selected from OH, SH, NH₂, C₁₋₃ alkylamino, and di(C₁₋₃ alkyl)amino;
L³ is C₁₋₆ alkylene; and
R⁵ is selected from C(═O)OH, S(═O)₂OH, and P(═O)(OH)₂.

In some embodiments, the compound of Formula (I) is not any one of the following compounds:

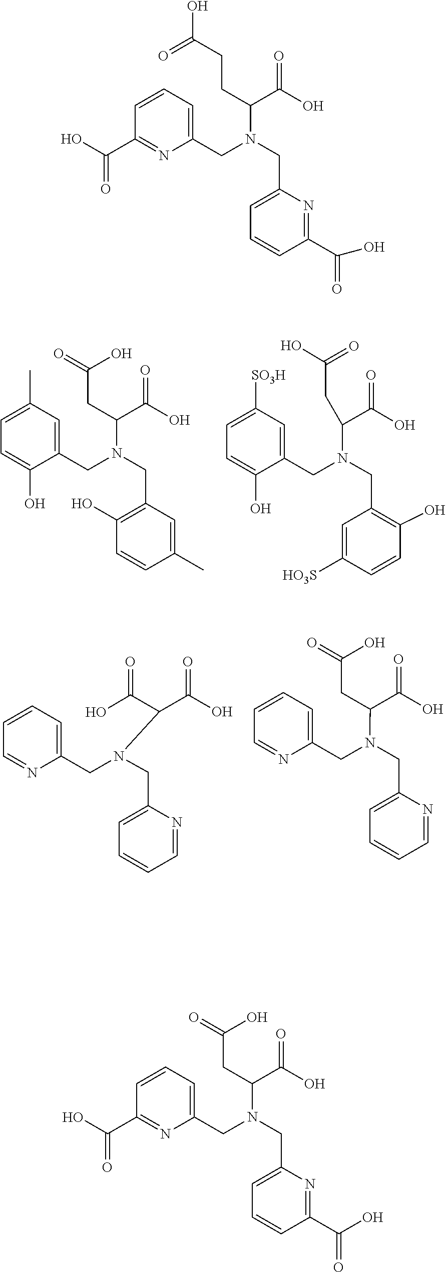

In some embodiments, L¹ is C₁₋₃ alkylene. In some embodiments, L¹ is selected from methylene, ethylene, and propylene. In some embodiments, L¹ is CH₂. In some embodiments, L¹ is CH₂CH₂. In some embodiments, L¹ is CH(CH₃).

In some embodiments, L² is C₁₋₃ alkylene. In some embodiments, L² is selected from methylene, ethylene, and propylene. In some embodiments, L² is CH₂. In some embodiments, L² is CH₂CH₂. In some embodiments, L² is CH(CH₃).

In some embodiments, L³ is C₁₋₆ alkylene (e.g., linear or branched). In some embodiments, L³ is selected from methylene, ethylene, n-propylene, isopropylene, n-butylene, isobutylene, sec-butylene, 2-methyl-1-butylene, n-pentylene, 3-pentylene, n-hexylene, and 1,2,2-trimethylpropylene. In some embodiments, L³ is CH₂CH₂.

In some embodiments:
L¹ is methylene;
L² is methylene; and
L³ is absent or selected from methylene, ethylene, and propylene.

In some embodiments:
L¹ is methylene;
L² is methylene; and
L³ is selected from propylene and butylene.

In some embodiments, each alkylene group herein is optionally substituted with 1, 2, 3, 4, or 5 independently selected R^{a}. In some embodiments, each R^{a} is independently selected from OH, NO₂, CN, halo, C₁₋₃ alkoxy, C₁₋₃ haloalkoxy, cyano-C₁₋₃ alkylene, HO—C₁₋₃ alkylene, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino. In some embodiments, each alkylene group herein is optionally substituted with 1, 2, or 3 independently selected R^{a}. In some embodiments, each alkylene group herein is optionally substituted with 1 or 2 independently selected R^{a}.

In some embodiments, R⁵ is C(═O)OH. In some embodiments, R⁵ is S(═O)₂OH. In some embodiments, R⁵ is P(═O)(OH)₂.

In some embodiments, X¹ is N. In some embodiments, X¹ is CR¹.

In some embodiments, R¹ is selected from OH, SH, and NH₂. In some embodiments, R¹ is OH. In some embodiments, R¹ is SH. In some embodiments, R¹ is NH₂.

In some embodiments, n is 0. In some embodiments, n is 1, 2, 3, or 4. In some embodiments, n is 1, 2, or 3. In some embodiments, n is 0, 1, or 2. In some embodiments, n is 0. In some embodiments, n is 1. In some embodiments, n is 2. In some embodiments, n is 3. In some embodiments, n is 4.

In some embodiments, each R³ is independently selected from OH, NO₂, CN, halo, C₁₋₃ alkyl, C₁₋₃ haloalkyl, C₁₋₃ alkoxy, C₁₋₃ haloalkoxy, cyano-C₁₋₃ alkylene, HO—C₁₋₃ alkylene, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, each R³ is independently selected from OH, NO₂, CN, halo, C₁₋₃ alkyl, C₁₋₃ haloalkyl, C₁₋₃ alkoxy, C₁₋₃ haloalkoxy, cyano-C₁₋₃ alkylene, HO—C₁₋₃ alkylene, amino, C₁₋₃ alkylamino, di(C₁₋₃ alkyl)amino, carboxy, and C₁₋₃ alkoxycarbonyl. In some embodiments, each R³ is independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, and carboxy.

In some embodiments, X² is N. In some embodiments, X¹ is CR².

In some embodiments, R² is selected from OH, SH, and NH₂. In some embodiments, R² is OH. In some embodiments, R² is SH. In some embodiments, R² is NH₂.

In some embodiments, m is 0. In some embodiments, m is 1, 2, 3, or 4. In some embodiments, m is 1, 2, or 3. In some embodiments, m is 0, 1, or 2. In some embodiments, m is 0. In some embodiments, m is 1. In some embodiments, m is 2. In some embodiments, m is 3. In some embodiments, m is 4.

In some embodiments, each R⁴ is independently selected from OH, NO₂, CN, halo, C₁₋₃ alkyl, C₁₋₃ haloalkyl, C₁₋₃ alkoxy, C₁₋₃ haloalkoxy, cyano-C₁₋₃ alkylene, HO—C₁₋₃ alkylene, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonylamino, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino.

In some embodiments, each R⁴ is independently selected from OH, NO₂, CN, halo, C₁₋₃ alkyl, C₁₋₃ haloalkyl, C₁₋₃ alkoxy, C₁₋₃ haloalkoxy, cyano-C₁₋₃ alkylene, HO—C₁₋₃ alkylene, amino, C₁₋₃ alkylamino, di(C₁₋₃ alkyl)amino, carboxy, and C₁₋₃ alkoxycarbonyl.

In some embodiments, each R⁴ is independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, and carboxy.

In some embodiments, n is 0 and m is 0.

In some embodiments, n is 1 or 2 and m is 1 or 2.

In some embodiments:
n is 1 or 2;
m is 1 or 2;
each R³ is independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, and carboxy; and
each R⁴ is independently selected from OH, NO₂, CN, halo, C₁₋₃ alkyl, C₁₋₃ haloalkyl, C₁₋₃ alkoxy, C₁₋₃ haloalkoxy, amino, C₁₋₃ alkylamino, di(C₁₋₃ alkyl)amino, and carboxy.

In some embodiments, X³ is selected from OH, SH, and NH₂. In some embodiments, X³ is selected from OH and SH. In some embodiments, X³ is OH. In some embodiments, X³ is SH. In some embodiments, X³ is NH₂. In some embodiments, X³ is C₁₋₃ alkylamino. In some embodiments, X³ is di(C₁₋₃ alkyl)amino.

In some embodiments, the compound of Formula (I) has formula:

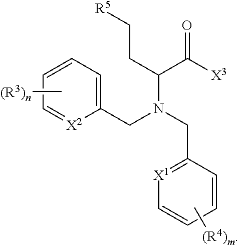

or a pharmaceutically acceptable salt thereof.

In some embodiments, the compound of Formula (I) has formula:

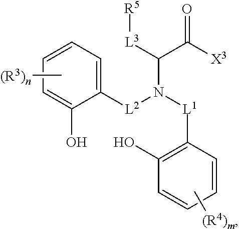

or a pharmaceutically acceptable salt thereof.

In some embodiments, the compound of Formula (I) has formula:

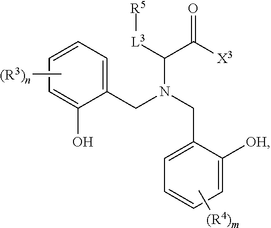

or a pharmaceutically acceptable salt thereof.

In some embodiments, the compound of Formula (I) has formula:

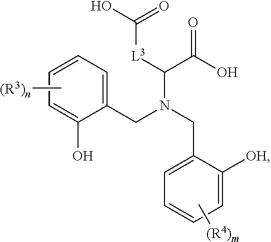

or a pharmaceutically acceptable salt thereof.

In some embodiments, the compound of Formula (I) has formula:

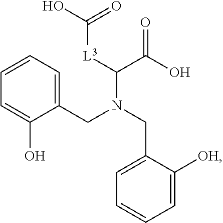

or a pharmaceutically acceptable salt thereof.

In some embodiments, the compound of Formula (I) has formula:

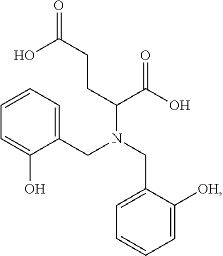 [SMILES: O=C(O)CCC(C(=O)O)N(Cc1ccccc1O)Cc1ccccc1O]

or a pharmaceutically acceptable salt thereof.

In some embodiments, the compound of Formula (I) has formula:

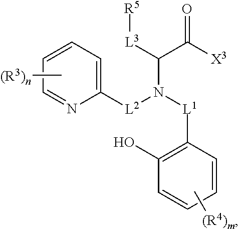

or a pharmaceutically acceptable salt thereof.

In some embodiments, the compound of Formula (I) has formula:

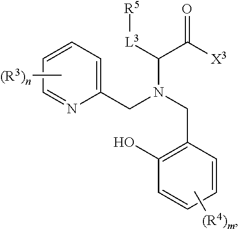

or a pharmaceutically acceptable salt thereof.

In some embodiments, the compound of Formula (I) has formula:

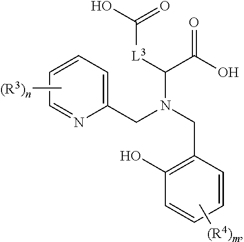

or a pharmaceutically acceptable salt thereof.

In some embodiments, the compound of Formula (I) has formula:

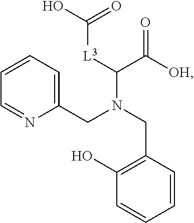

or a pharmaceutically acceptable salt thereof.

In some embodiments, the compound of Formula (I) has formula:

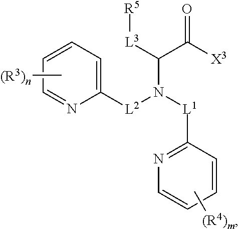

or a pharmaceutically acceptable salt thereof.

In some embodiments, the compound of Formula (I) has formula:

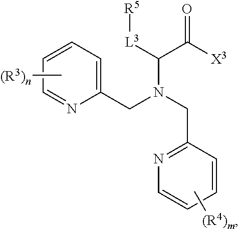

or a pharmaceutically acceptable salt thereof.

In some embodiments, the compound of Formula (I) has formula:

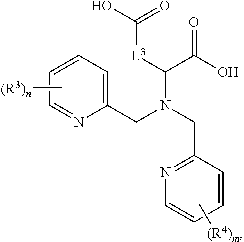

or a pharmaceutically acceptable salt thereof.

In some embodiments, the compound of Formula (I) has formula:

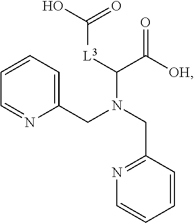

or a pharmaceutically acceptable salt thereof.

In some embodiments, the compound of Formula (I) is any one of the following compounds:

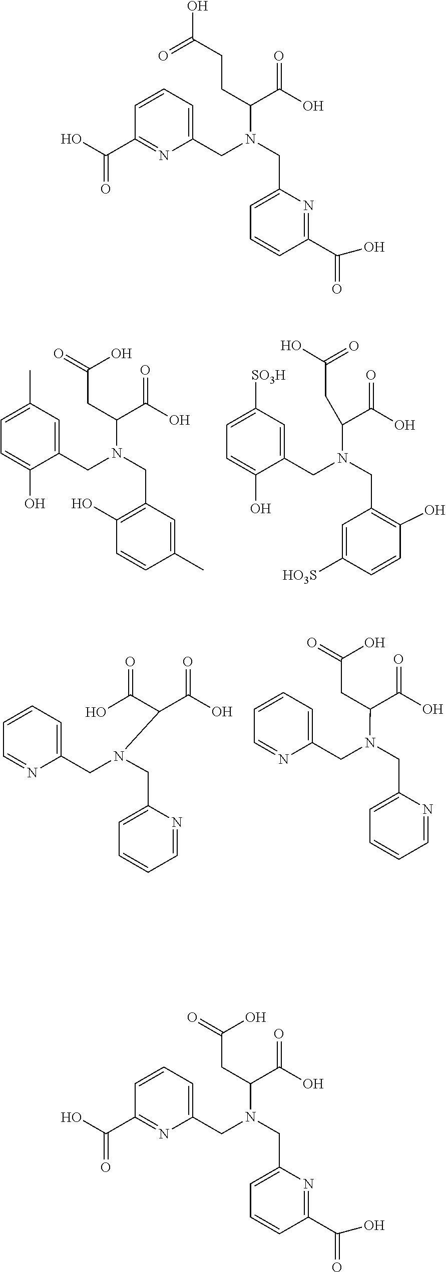

or a pharmaceutically acceptable salt thereof.

### Compounds of Formula (B)

In some embodiments, the present disclosure provides a compound of Formula (B):

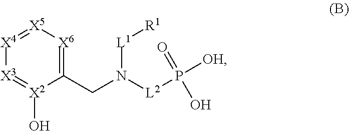

or a salt (e.g., pharmaceutically acceptable salt) thereof, wherein:
L¹ is —C₁₋₃ alkylene-, optionally substituted with R¹⁰;
L² is —C₁₋₃ alkylene-, optionally substituted with R¹⁰;
each R¹⁰ is independently —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, or —(C₁₋₃ alkyl)_{q}(C═O)NHSO₂R⁸;
R¹ is selected from CO₂R^{A}, P(O)(OR^{A})(OR^{B}), and (C═O)NR^{A}R¹⁰;
each q is independently 0 or 1;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N; R³, R¹, R⁵, and R⁶ and are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN.

In some embodiments, if R¹ is P(O)(OH)(OH), then R⁵ is not C₁₋₃ alkyl or halo. In some embodiments, R⁵ is not C₁₋₃ alkyl. In some embodiments, R⁵ is not halo.

In some embodiments, the compound of Formula (B) is not any one of the following compounds:

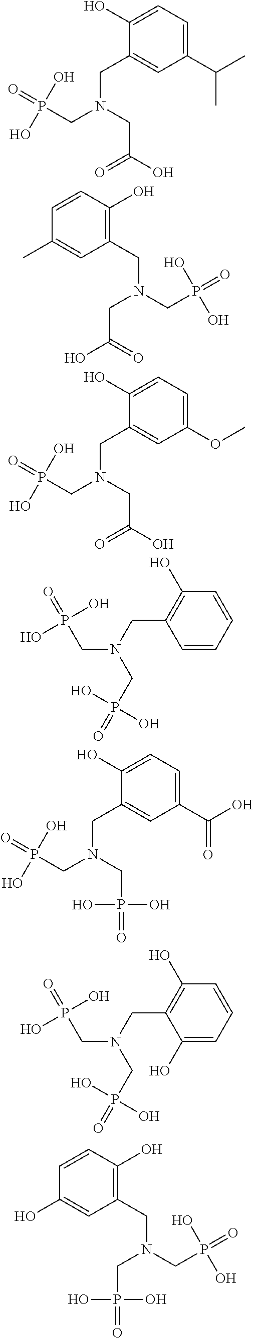

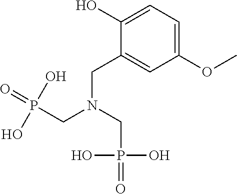 [SMILES: COc1ccc(O)c(CN(CP(=O)(O)O)CP(=O)(O)O)c1]

In some embodiments, X², X³, X⁴, X³, X⁶, L¹, and R¹ are as described herein for Formula (A). In some embodiments, L² is as described herein for L¹ in Formula (A). In some embodiments, L² is —C₁₋₃ alkylene-. In some embodiments, L² is —C₁₋₃ alkylene-substituted with R¹⁰. In some embodiments, L² is selected from methylene and ethylene. In some embodiments, L¹ is —C₁₋₃ alkylene-. In some embodiments, L¹ is —C₁₋₃ alkylene-substituted with R¹⁰. In some embodiments, L¹ is ethylene.

In some embodiments, the compound has formula:

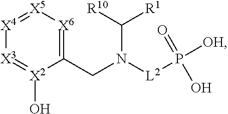

or a pharmaceutically acceptable salt thereof.

In some embodiments, the compound has formula:

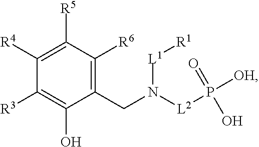

or a pharmaceutically acceptable salt thereof;
wherein R⁵ is selected from C₁₋₃ alkoxy, C₁₋₃ haloalkyl, C₁₋₃ haloalkoxy, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸.

In some embodiments, the compound has formula:

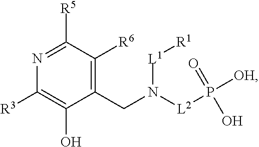

or a pharmaceutically acceptable salt thereof.

In some embodiments, R¹⁰ is selected from —(C₁₋₃ alkyl)_{q}CO₂R^{A}, —(C₁₋₃ alkyl)_{q}OP(R⁸)O₂R^{B}, —(C₁₋₃ alkyl), (C═O)NHSO₂R⁸, and —(C₁₋₃ alkyl)_{q}PO₃R^{A}R⁸.

In some embodiments, R¹⁰ is —(C₁₋₃ alkyl)_{q}CO₂R^{A}. In some embodiments, R¹⁰ is —(C₁₋₃ alkyl)_{q}PO₃R^{A}R⁸. In some embodiments, q is 0. In some embodiments, q is 1.

In some embodiments, the compound is selected from any one of the following compounds:
or a salt thereof.

In some embodiments, the compound of Formula (B) is selected from any one of the following compounds:

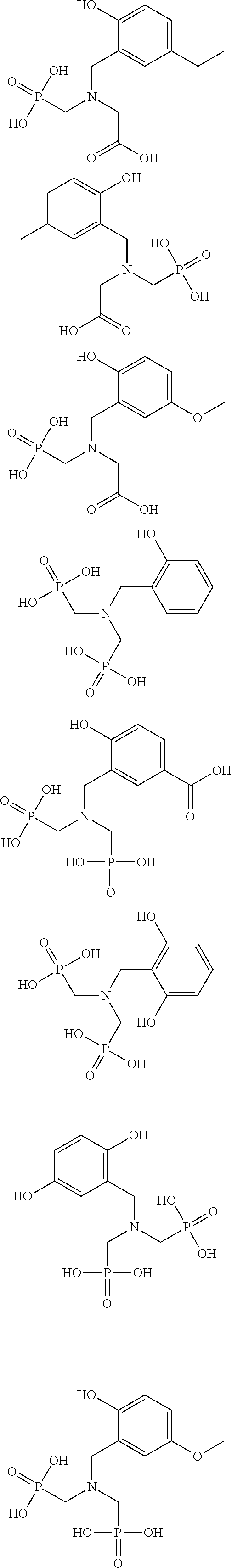

or a salt thereof.

### Compounds of Formula (C)

In some embodiments, the present disclosure provides a compound of Formula (C):

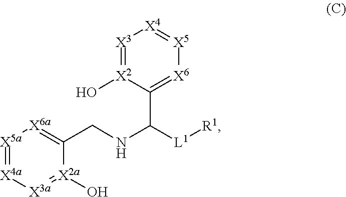

or a salt (e.g., pharmaceutically acceptable salt) thereof, wherein:
L¹ is —C₁₋₃ alkylene-, or L¹ is absent;
R¹ is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
X^{2a} is selected from N and C;
X^{3a} is selected from N and CR^{3a};
X^{4a} is selected from N and CR^{4a};
X^{5a} is selected from N and CR^{5a};
X^{6a} is selected from N and CR^{6a};
provided that no more than two of X^{2a}, X^{3a}, X^{4a}, X^{5a}, and X^{6a} are N;
R³, R^{3a}, R⁴, R^{4a}, R⁵, R^{5a}, R⁶, and R^{6a} are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸;
each q is independently 0 or 1;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN.

In some embodiments, X², X³, X⁴, X⁵, X⁶, X^{2a}, X^{3a}, X^{4a}, X^{5a}, X⁶a, L¹, and R¹ are as described herein for Formula (A) or (B).

In some embodiments, L¹ is absent. In some embodiments, L¹ is —C₁₋₃ alkylene-. In some embodiments, L¹ is —C₁₋₃ alkylene-substituted with R¹⁰. In some embodiments, L¹ is —C₁₋₃ alkylene-substituted with CH₂OH, C(O)OH, SO₃H, or OP(O)(OH)₂. In some embodiments, L¹ is —C₁₋₃ alkylene-substituted with C₁₋₃ alkyl-OPO(OH)(OH), —C₁₋₃ alkyl-C(O)(OH), —C₁₋₃ alkyl-SO₂ (OH), or —C₁₋₃ alkyl-(C═O)NH₂.

In some embodiments, L¹ is selected from methylene, ethylene, and propylene.

In some embodiments, the compound has formula:

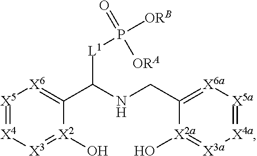

or a pharmaceutically acceptable salt thereof.

In some embodiments, the compound has formula:

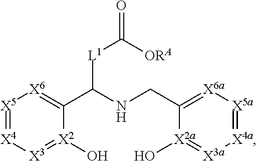

or a pharmaceutically acceptable salt thereof.

In some embodiments, the compound has formula:

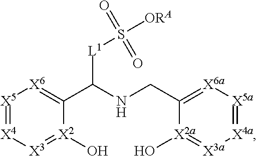

or a pharmaceutically acceptable salt thereof.

In some embodiments, the compound has formula:

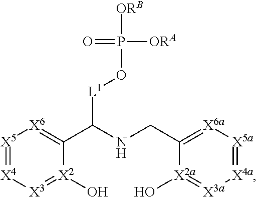

or a pharmaceutically acceptable salt thereof.

In some embodiments, the compound has formula:

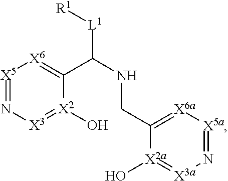

or a pharmaceutically acceptable salt thereof.

In some embodiments, the compound has formula:

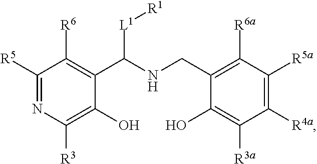

or a pharmaceutically acceptable salt thereof.

In some embodiments, the compound has formula:

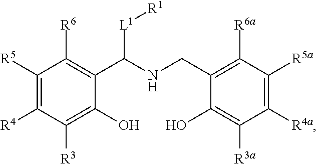

or a pharmaceutically acceptable salt thereof.

In some embodiments, at least one of R³, R^{3a}, R⁴, R^{4a}, R⁵, R^{5a}, R⁶, and R^{6a} is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl), (C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸.

In some embodiments, at least one of R³, R^{3a}, R⁴, R^{4a}, R⁵, R^{5a}, R⁶, and R^{6a} is selected from C₁₋₃ alkyl, —(C₁₋₃ alkyl)_{q}OH, —(C₁₋₃ alkyl)_{q}SO₃R^{A}, —(C₁₋₃ alkyl)_{q}CO₂R^{A}, —(C₁-3 alkyl)_{q}OPO₃R^{A}R^{B}, and —(C₁₋₃ alkyl)_{q}PO₃R^{A}R⁸.

In some embodiments, at least one of R³, R^{3a}, R⁴, R^{4a}, R⁵, R^{5a}, R⁶, and R^{6a} is selected from C₁₋₃ alkyl, CH₂OH, C(O)OH, SO₃H, and CH₂OP(O)(OH)₂. In some embodiments, at least one, at least two, or at least three of R³, R^{3a}, R⁴, R^{4a}, R⁵, R^{5a}, R⁶, and R^{6a} are independently selected from C₁₋₃ alkyl-OPO(OR^{A})(OR^{B}), —C₁₋₃ alkyl-CO₂R^{A}, —C₁₋₃ alkyl-SO₃R^{A}, and —C₁₋₃ alkyl-(C═O)NR^{A}R^{B}. In some embodiments, at least one, at least two, or at least three of R³, R^{3a}, R⁴, R^{4a}, R⁵, R^{5a}, R⁶, and R^{6a} are independently selected from C₁₋₃ alkyl-OPO(OH)(OH), —C₁₋₃ alkyl-C(O)(OH), —C₁₋₃ alkyl-SO₂ (OR^{A}), and —C₁₋₃ alkyl-(C═O)NH₂.

In some embodiments, q is 0. In some embodiments, q is 1.

In some embodiments, the compound of Formula (C) is selected from any one of the following compounds:

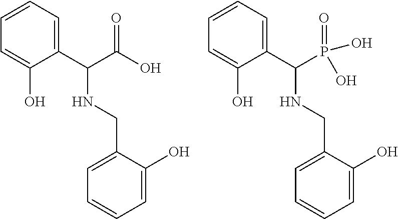 [SMILES: O=C(O)C(NCc1ccccc1O)c1ccccc1O ; O=P(O)(O)C(NCc1ccccc1O)c1ccccc1O]

or a salt thereof.

### Compounds of Formula (D)

In some embodiments, the present disclosure provides a compound of Formula (D):

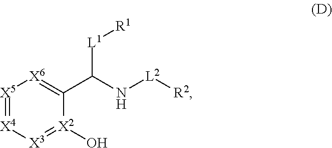

or a salt (e.g., pharmaceutically acceptable salt) thereof, wherein:
L¹ is —C₁₋₃ alkylene-, or L¹ is absent;
L² is —C₁₋₃ alkylene-;
R¹ is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
R² is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
provided that at least one of R¹ and R² is P(O)(OR^{A})(OR⁸);
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
R³, R⁴, R⁵, and R⁶ and are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN.

In some embodiments, the compound of Formula (D) is not:

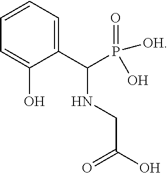 [SMILES: O=C(O)CNC(c1ccccc1O)P(=O)(O)O]

In some embodiments, X², X³, X⁴, X⁵, X⁶, L¹, L², R¹, and R² in Formula (D) are as described herein for Formulae (A), (B), or (C). In some embodiments, L¹ is absent. In some embodiments, L¹ is —C₁₋₃ alkylene-. In some embodiments, L¹ is selected from methylene, ethylene, and propylene. In some embodiments, L² is —C₁₋₃ alkylene-. In some embodiments, L² is selected from methylene, ethylene, and propylene.

In some embodiments, the compound has formula:

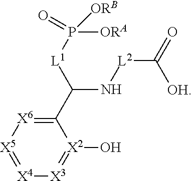

or a pharmaceutically acceptable salt thereof.

In some embodiments, the compound has formula:

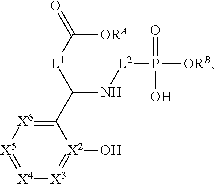

or a pharmaceutically acceptable salt thereof.

In some embodiments, the compound has formula:

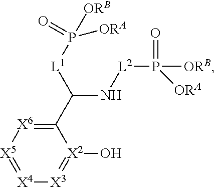

or a pharmaceutically acceptable salt thereof.

In some embodiments, the compound has formula:

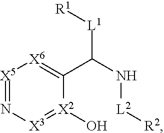

or a pharmaceutically acceptable salt thereof.

In some embodiments, the compound has formula:

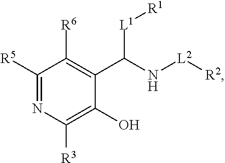

or a pharmaceutically acceptable salt thereof.

In some embodiments, the compound has formula:

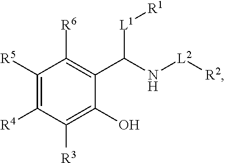

or a pharmaceutically acceptable salt thereof.

In some embodiments, at least one of R³, R⁴, R⁵, and R⁶ is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸.

In some embodiments, at least one of R³, R^{3a}, R⁴, R^{4a}, R⁵, R^{5a}, R⁶, and R^{6a} is selected from C₁₋₃ alkyl, —(C₁₋₃ alkyl)_{q}OH, —(C₁₋₃ alkyl)_{q}SO₃R^{A}, —(C₁₋₃ alkyl)_{q}CO₂R^{A}, —(C₁-3 alkyl)_{q}OPO₃R^{A}R^{B}, and —(C₁₋₃ alkyl)_{q}PO₃R^{A}R⁸.

In some embodiments, at least one of R³, R^{3a}, R⁴, R^{4a}, R⁵, R^{5a}, R⁶, and R^{6a} is selected from C₁₋₃ alkyl, CH₂OH, C(O)OH, SO₃H, and CH₂OP(O)(OH)₂.

In some embodiments, q is 0. In some embodiments, q is 1.

In some embodiments, the compound is selected from any one of the following

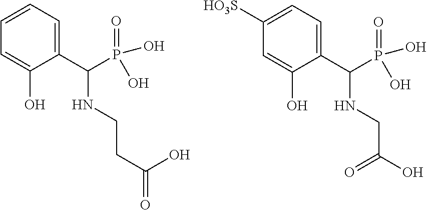 [SMILES: O=C(O)CCNC(c1ccccc1O)P(=O)(O)O ; O=C(O)CNC(c1ccc(S(=O)(=O)O)cc1O)P(=O)(O)O]

or a salt thereof.

In some embodiments, the compound of Formula (D) is:

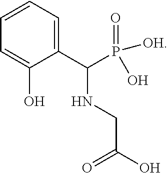 [SMILES: O=C(O)CNC(c1ccccc1O)P(=O)(O)O]

or a salt thereof.

### Iron Complexes

In some embodiments, the present application provides a complex of Fe³⁺ ion and a compound of any of Formulae described herein, or a pharmaceutically acceptable salt thereof. In some embodiments, the present application provides a complex of Fe²⁺ ion and a compound of any of Formulae disclosed herein, or a pharmaceutically acceptable salt thereof. In some embodiments, the present application provides a complex that does not comprise (or is substantially free from) Fe²⁺ ion.

In some embodiments, the compound binds Fe³⁺ with an affinity that is lower than affinity of Fe³⁺ to transferrin protein (log K_{cond} at pH 7.4 of about 20.7 and 19.4 for the first and second binding sites, respectively). In some embodiments, affinity of Fe³⁺ to the transferrin is about 1.05×, about 10×, about 100×, about 1000×, or about 10,000× greater compared to binding affinity to iron for the compounds within the instant claims. In some embodiments, the compound comprises a thermodynamically stable Fe(III) complex that also binds Fe³⁺ with about 10×, about 100×, about 1000×, or about 10,000× greater affinity than other Fe chelators found in the body, such as citrate, acetate lactate, phosphate, or carbonate. Without being bound by any theory, it is believed that the thermodynamic stability of the complex minimizes concentrations of unchelated and dissociated “labile” Fe in the bloodstream upon administration. In some embodiments, the complexes within the present claims are kinetically labile. Without being bound by any theory or speculation, it is believed that the kinetic lability of the complex enable rapid delivery of the Fe³⁺ to the transferrin protein. In some embodiments, the compound binds Fe³⁺ selectively when compared to other metal ions commonly present in the bloodstream, such as divalent metal ions like Zn²⁺. For example, the compound is about 1,000×, about 500×, about 200×, about 150×, about 100×, about 50×, about 10×, or about 2× more selective toward binding Fe³⁺ over any divalent metal of the serum, or any combination thereof. Without being bound by any theory, it is believed that low affinity of the compounds of this disclosure to divalent ions such as Zn²⁺ allows to preserve serum metal ion concentrations and avoids redistributing endogenous divalent plasma metals upon administration of the compound (iron complex) to anemic patient. In some embodiments, the Fe(III) within the complex resist reduction to Fe(II) and adventitious redox cycling. In some embodiments, the complexes do not penetrate cellular membranes and substantially remain in extracellular fluid (such as blood and lymph) where transferrin is present. In some embodiments, the compounds described herein are eliminated and excreted partially through hepatobiliary pathway. For example, at least 10 wt. %, at least 20 wt. %, at least 30 wt. %, at least 40 wt. %, at least 50 wt. %, at least 60 wt. %, at least 75 wt. %, or at least 90 wt. % of the compound is excreted through hepatobiliary pathway. Without being bound by any particular theory or speculation, it is believed that the hepatobiliary elimination ensures efficient elimination of the compound in patients with advanced kidney disease.

### Additional Ligands

In some embodiments, the coordination complex further comprises at least one additional iron ligand, in addition to the ligand compound of Formula (A) or Formula (I). In some embodiments, each additional iron ligand is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof. In some embodiments, each additional iron ligand is a group L as described hereinbelow.

In some embodiments, each L is independently selected from H₂O and NH₃. In some embodiments, each L is H₂O. In some embodiments, L is an inorganic anion such as Cl, Br, SO₄, HCO₃, CO₃, PO₄, nitrate, nitrite, or the like. In some embodiments, each L is an acid or a base commonly present in the plasma. Examples of such L include organic acids such as citric acid, tartaric acid, ascorbic acid, malic acid, succinic acid, or acetic acid. In some embodiments, L is a sugar such as glucose, fructose, mannose, or galactose. In some embodiments, any two L may be joined together to form a single iron ligand. In some embodiments, L is an acid or a base described herein in the “pharmaceutically acceptable salts” section, or an anion or a cation thereof.

Iron Complex with Ligand of Formula (A)

In some embodiments, the present disclosure provides a complex of Fe³⁺ ion and a compound of Formula (A) as described herein, having formula:

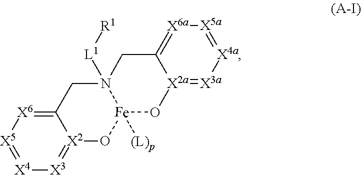

or a pharmaceutically acceptable salt thereof, wherein:
[symbol] indicates a coordinate bond;
each L is independently a Fe³⁺ ligand;
p is 0, 1 or 2; and
X², X³, X⁴, X⁵, X⁶, X^{2a}, X^{3a}, X^{4a}, X^{5a}, X⁶a, L¹, and R¹ are as described herein for Formula (A). In some embodiments, the iron complex is useful in any of the pharmaceutical formulations and dosage forms for treating diseases described herein, such as iron deficiency anemia.

In some embodiments:
L¹ is —C₁₋₃ alkylene-, optionally substituted with R¹⁰;
each R¹⁰ is independently —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, or —(C₁₋₃ alkyl)_{q}(C═O)NHSO₂R⁸;
R¹ is selected from C(O)O, P(O)(O)(OR^{B}), and (C═O)NR¹⁰;
each q is independently 0 or 1;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
X^{2a} is selected from N and C;
X^{3a} is selected from N and CR^{3a};
X^{4a} is selected from N and CR^{4a};
X^{5a} is selected from N and CR^{5a};
X^{6a} is selected from N and CR^{6a};
provided that no more than two of X^{2a}, X^{3a}, X^{4a}, X^{5a}, and X^{6a} are N;
R³, R³⁴, R⁴, R^{4a}, R⁵, R^{5a}, R⁶, and R^{6a} are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN,
provided that if L¹-R¹ is CH₂C(O)O or CH₂CH₂C(O)O, then R⁵ and R^{5a} are not both S(O)₂OH, methyl, halo, or tert-butyl.

In some embodiments, the compound has formula:

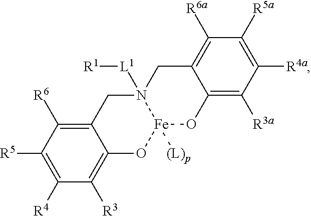

or a pharmaceutically acceptable salt thereof.

In some embodiments, the compound has formula:

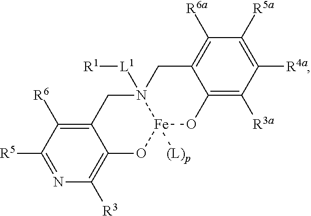

or a pharmaceutically acceptable salt thereof.

In some embodiments, the compound has formula:

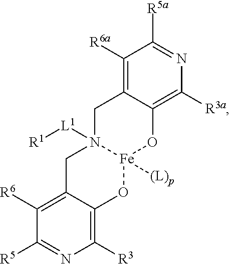

or a pharmaceutically acceptable salt thereof.

In some embodiments, L¹ is —C₁₋₃ alkylene-. In some embodiments, L¹ is —C₁₋₃ alkylene-substituted with R¹⁰. In some embodiments, L¹ is selected from methylene, ethylene, and propylene.

In some embodiments, the compound has formula:

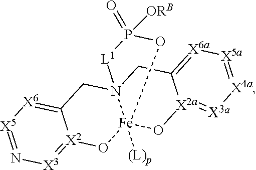

or a pharmaceutically acceptable salt thereof.

In some embodiments, the compound has formula:

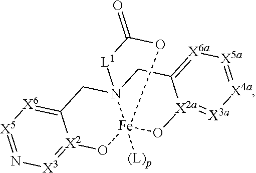

or a pharmaceutically acceptable salt thereof.

In some embodiments, the compound has formula:

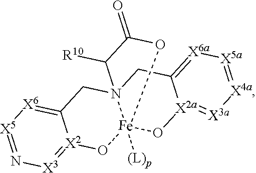

or a pharmaceutically acceptable salt thereof.

In some embodiments, the compound has formula:

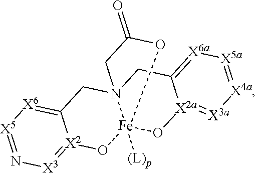

or a pharmaceutically acceptable salt thereof.

In some embodiments, the compound has formula:

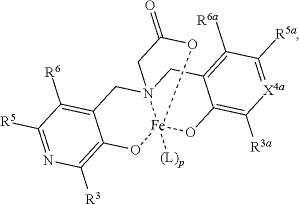

or a pharmaceutically acceptable salt thereof.

In some embodiments, the compound has formula:

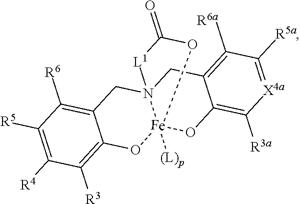

or a pharmaceutically acceptable salt thereof, wherein:
L¹ is selected from methylene and ethylene,
R^{5a} is H; and
R⁵ is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰.

In some embodiments, at least one of R³, R^{3a}, R⁴, R^{4a}, R⁵, R^{5a}, R⁶, and R^{6a} is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰. In some embodiments, at least one of R³, R^{3a}, R⁴, R^{4a}, R⁵, R^{5a}, R⁶, and R^{6a} is R¹⁰.

In some embodiments, at least one, at least two, or at least three of R³, R^{3a}, R⁴, R^{4a}, R⁵, R^{5a}, R⁶, and R^{6a} are each independently selected from —(C₁₋₃ alkyl)_{q}CO₂R^{A}, —(C₁₋₃ alkyl)_{q}OP(R⁸)O₂R^{B}, —(C₁₋₃ alkyl), (C═O)NHSO₂R⁸, and —(C₁₋₃ alkyl)_{q}PO₃R^{A}R⁸. In some embodiments, at least one, at least two, or at least three of R³, R^{3a}, R⁴, R^{4a}, R⁵, R^{5a}, R⁶, and R^{6a} are independently selected from C₁₋₃ alkyl-OPO(OR^{A})(OR^{B}), —C₁₋₃ alkyl-CO₂R^{A}, —C₁₋₃ alkyl-SO₃R^{A}, and —C₁₋₃ alkyl-(C═O)NR^{A}R^{B}. In some embodiments, at least one, at least two, or at least three of R³, R^{3a}, R⁴, R^{4a}, R⁵, R^{5a}, R⁶, and R^{6a} are independently selected from C₁₋₃ alkyl-OPO(OH)(OH), —C₁₋₃ alkyl-C(O)(OH), —C₁₋₃ alkyl-SO₂ (OH), and —C₁₋₃ alkyl-(C═O)NH₂.

In some embodiments, R¹⁰ is selected from —(C₁₋₃ alkyl)_{q}CO₂R^{A}, —(C₁₋₃ alkyl)_{q}OP(R⁸)O₂R^{B}, —(C₁₋₃ alkyl). (C═O)NHSO₂R⁸, and —(C₁₋₃ alkyl)_{q}PO₃R^{A}R⁸. In some embodiments, R¹⁰ is —(C₁₋₃ alkyl)_{q}CO₂R^{A}. In some embodiments, q is 0. In some embodiments, q is 1.

In some embodiments, each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.

In some embodiments, the compound has formula:

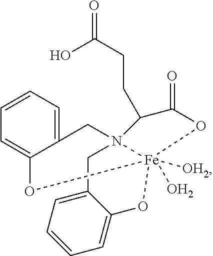

or a pharmaceutically acceptable salt thereof.
   Iron Complex with Ligand of Formula (I)

In some embodiments, the present application provides a complex of Fe³⁺ ion and a compound of Formula (I) as described herein. In some embodiments, the complex has a formula selected from any one of the following:

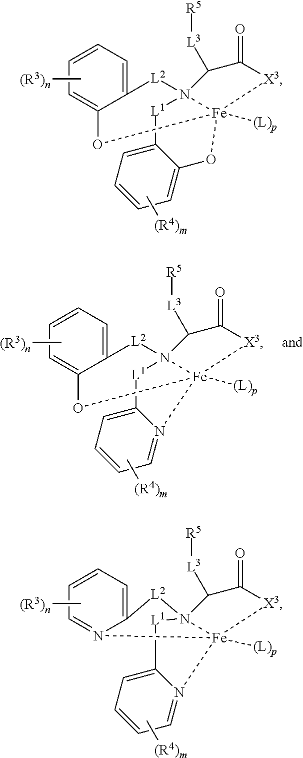

or a pharmaceutically acceptable salt thereof, wherein:
each [symbol] represents a coordination bond;
each L is independently an iron ligand; and
p is 0, 1 or 2.

In some embodiments, p is 0. In some embodiments, p is 1. In some embodiments, p is 2.

In some embodiments, the present application provides a complex of Fe³⁺ ion and a compound of Formula (I), having formula:

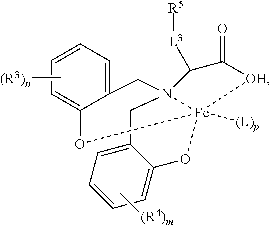

or a pharmaceutically acceptable salt thereof.

In some embodiments, the present application provides a complex of Fe³⁺ ion and a compound of Formula (I), having formula:

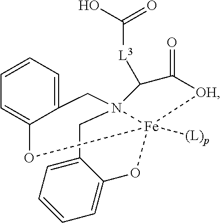

or a pharmaceutically acceptable salt thereof.

In some embodiments, the present application provides a complex of Fe³⁺ ion and a compound of Formula (I), having formula:

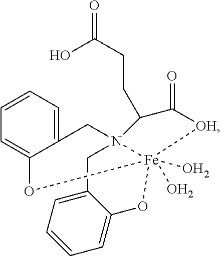

or a pharmaceutically acceptable salt thereof.

In some embodiments, the present application provides a complex of Fe³⁺ ion and a compound of Formula (I), having formula:

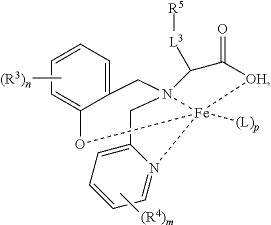

or a pharmaceutically acceptable salt thereof.

In some embodiments, the present application provides a complex of Fe³⁺ ion and a compound of Formula (I), having formula:

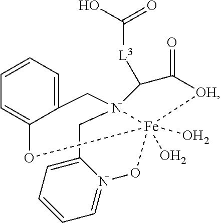

or a pharmaceutically acceptable salt thereof.

In some embodiments, the present application provides a complex of Fe³⁺ ion and a compound of Formula (I), having formula:

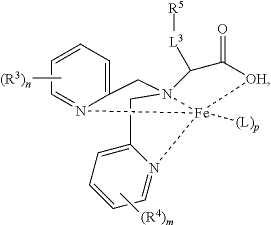

or a pharmaceutically acceptable salt thereof.

In some embodiments, the present application provides a complex of Fe³⁺ ion and a compound of Formula (I), having formula:

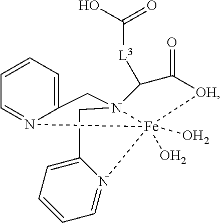

or a pharmaceutically acceptable salt thereof.
   Iron Complex with Ligand of Formula (B)

In some embodiments, the present application provides a complex of Fe³⁺ ion and a compound of Formula (B) as described herein. In some embodiments, the complex has a formula:

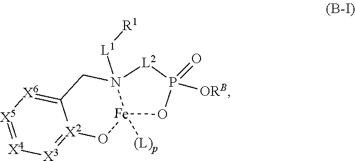

or a pharmaceutically acceptable salt thereof, wherein:
[symbol] indicates a coordinate bond;
each L is independently a Fe³⁺ ligand;
p is 0, 1 or 2; and
X², X³, X⁴, X⁵, X⁶, L¹, L², R¹, and R^{B} are as described herein for Formula (B).

In some embodiments:
L¹ is —C₁₋₃ alkylene-, optionally substituted with R¹⁰;
L² is —C₁₋₃ alkylene-, optionally substituted with R¹⁰;
each R¹⁰ is independently —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, or —(C₁₋₃ alkyl)_{q}(C═O)NHSO₂R⁸;
R¹ is selected from CO₂R^{A}, P(O)(OR^{A})(OR^{B}), and (C═O)NR^{A}R¹⁰;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
R³, R⁴, R⁵, and R⁶ and are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰; each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN.

In some embodiments, L² is —C₁₋₃ alkylene-. In some embodiments, L² is —C₁₋₃ alkylene-substituted with R¹⁰. In some embodiments, L² is selected from methylene and ethylene. In some embodiments, L¹ is —C₁₋₃ alkylene-. In some embodiments, L¹ is —C₁₋₃ alkylene-substituted with R¹⁰.

In some embodiments, L¹ is selected from methylene and ethylene. In some embodiments, R¹ is (C═O)NR^{A}R¹⁰.

In some embodiments, the compound has formula:

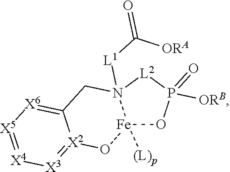

or pharmaceutically acceptable salt thereof.

In some embodiments, the compound has formula:

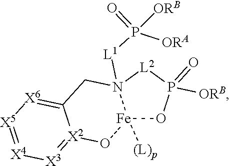

or pharmaceutically acceptable salt thereof.

In some embodiments, the compound has formula:

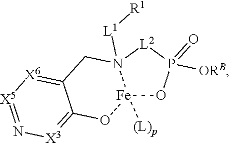

or pharmaceutically acceptable salt thereof.

In some embodiments, the compound has formula:

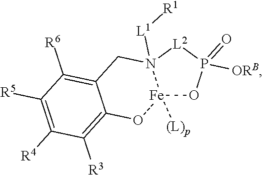

or pharmaceutically acceptable salt thereof.

In some embodiments, at least one of R³, R⁴, R⁵, and R⁶ is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰. In some embodiments, at least one of R³, R⁴, R⁵, and R⁶ is R¹⁰.

In some embodiments, at least one, at least two, or at least three of R³, R⁴, R⁵, and R⁶, and are each independently selected from —(C₁₋₃ alkyl)_{q}CO₂R^{A}, —(C₁₋₃ alkyl)_{q}OP(R⁸)O₂R^{B}, —(C₁₋₃ alkyl)_{q}(C═O)NHSO₂R⁸, and —(C₁₋₃ alkyl)_{q}PO₃R^{A}R⁸. In some embodiments, at least one, at least two, or at least three of R³, R⁴, R⁵, and R⁶ are independently selected from C₁₋₃ alkyl-OPO(OR^{A})(OR^{B}), —C₁₋₃ alkyl-CO₂R^{A}, —C₁₋₃ alkyl-SO₃R^{A}, and —C₁₋₃ alkyl-(C═O)NR^{A}R^{B}. In some embodiments, at least one, at least two, or at least three of R³, R⁴, R⁵, and R⁶ are independently selected from C₁₋₃ alkyl-OPO(OH)(OH), —C₁₋₃ alkyl-C(O)(OH), —C₁₋₃ alkyl-SO₂ (OH), and —C₁₋₃ alkyl-(C═O)NH₂.

In some embodiments, R¹⁰ is selected from —(C₁₋₃ alkyl)_{q}CO₂R^{A}, —(C₁₋₃ alkyl)_{q}OP(R⁸)O₂R^{B}, —(C₁₋₃ alkyl), (C═O)NHSO₂R⁸, and —(C₁₋₃ alkyl)_{q}PO₃R^{A}R⁸. In some embodiments, R¹⁰ is —(C₁₋₃ alkyl)_{q}CO₂R^{A}.

In some embodiments, q is 0. In some embodiments, q is 1.

In some embodiments, each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.

Iron Complex with Ligand of Formula (C)

In some embodiments, the present application provides a complex of Fe³⁺ ion and a compound of Formula (C) as described herein. In some embodiments, the complex has a formula:

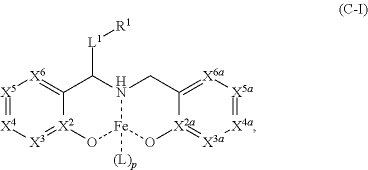

or a pharmaceutically acceptable salt thereof, wherein:
[symbol] indicates a coordinate bond;
each L is independently a Fe³⁺ ligand;
p is 0, 1 or 2; and
X², X³, X⁴, X⁵, X⁶, L¹, R¹, X^{2a}, X^{3a}, X^{4a}, X^{5a}, and X^{6a} are as described herein for Formula (C).

In some embodiments:
L¹ is —C₁₋₃ alkylene-, or L¹ is absent;
R¹ is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
X^{2a} is selected from N and C;
X^{3a} is selected from N and CR^{3a};
X^{4a} is selected from N and CR^{4a};
X^{5a} is selected from N and CR^{5a};
X^{6a} is selected from N and CR^{6a};
provided that no more than two of X^{2a}, X^{3a}, X^{4a}, X^{5a}, and X^{6a} are N;
R³, R^{3a}, R⁴, R^{4a}, R⁵, R^{5a}, R⁶, and R^{6a} are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl), (C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸;
each q is independently 0 or 1;
each R⁸ is selected from C_{3.9} alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁-3 haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN.

In some embodiments, L¹ is absent. In some embodiments, L¹ is —C₁₋₃ alkylene-. In some embodiments, L¹ is —C₁₋₃ alkylene-substituted with R¹⁰. In some embodiments, L¹ is selected from methylene, ethylene, and propylene.

In some embodiments, the compound has formula:

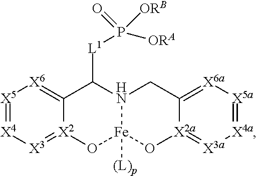

or a pharmaceutically acceptable salt thereof.

In some embodiments, the compound has formula:

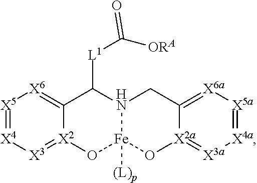

or a pharmaceutically acceptable salt thereof.

In some embodiments, the compound has formula:

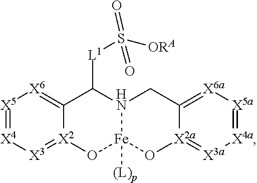

or a pharmaceutically acceptable salt thereof.

In some embodiments, the compound has formula:

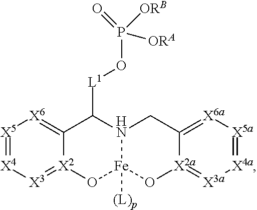

or a pharmaceutically acceptable salt thereof.

In some embodiments, the compound has formula:

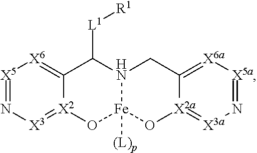

or a pharmaceutically acceptable salt thereof.

In some embodiments, the compound has formula:

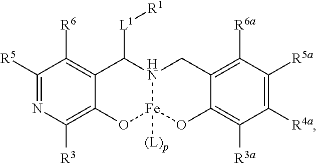

or a pharmaceutically acceptable salt thereof.

In some embodiments, the compound has formula:

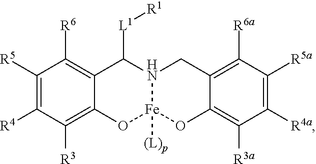

or a pharmaceutically acceptable salt thereof.

In some embodiments, at least one of R³, R^{3a}, R¹, R^{4a}, R⁵, R^{5a}, R⁶, and R^{6a} is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl), (C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸.

In some embodiments, at least one of R³, R^{3a}, R⁴, R^{4a}, R⁵, R^{5a}, R⁶, and R^{6a} is selected from C₁₋₃ alkyl, —(C₁₋₃ alkyl)_{q}OH, —(C₁₋₃ alkyl)_{q}SO₃R^{A}, —(C₁₋₃ alkyl)_{q}CO₂R^{A}, —(C₁₋₃ alkyl)_{q}OPO₃R^{A}R^{B}, and —(C₁₋₃ alkyl)_{q}PO₃R^{A}R⁸.

In some embodiments, at least one of R³, R^{3a}, R⁴, R^{4a}, R⁵, R^{5a}, R⁶, and R^{6a} is selected from C₁₋₃ alkyl, CH₂OH, C(O)OH, SO₃H, and CH₂OP(O)(OH)₂.

In some embodiments, q is 0. In some embodiments, q is 1.

In some embodiments, each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.

Iron Complex with Ligand of Formula (D)

In some embodiments, the present application provides a complex of Fe³⁺ ion and a compound of Formula (D) as described herein. In some embodiments, the complex has a formula:

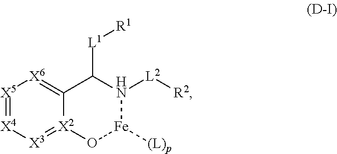

or a pharmaceutically acceptable salt thereof, wherein:
[symbol] indicates a coordinate bond;
each L is independently a Fe³⁺ ligand;
p is 0, 1 or 2; and
X², X³, X⁴, X³, X⁶, L¹, R¹, L², and R² are as described herein for Formula (D).

In some embodiments:
L¹ is —C₁₋₃ alkylene-, or L¹ is absent;
L² is —C₁₋₃ alkylene-;
R¹ is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
R² is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
R³, R⁴, R⁵, and R⁶ and are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl). (C═O)NHSO₂R⁸;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN.

In some embodiments, the compound is not any one of the following compounds:

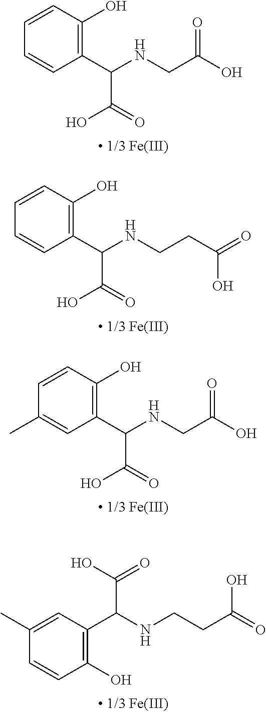 [SMILES: O=C(O)CNC(C(=O)O)c1ccccc1O ; O=C(O)CCNC(C(=O)O)c1ccccc1O ; Cc1ccc(O)c(C(NCC(=O)O)C(=O)O)c1 ; Cc1ccc(O)c(C(NCCC(=O)O)C(=O)O)c1]

In some embodiments, L¹ is absent. In some embodiments, L¹ is —C₁₋₃ alkylene-. In some embodiments, L¹ is selected from methylene, ethylene, and propylene. In some embodiments, L² is —C₁₋₃ alkylene-. In some embodiments, L² is selected from methylene, ethylene, and propylene.

In some embodiments, the compound has formula:

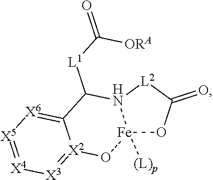

or a pharmaceutically acceptable salt thereof.

In some embodiments, the compound has formula:

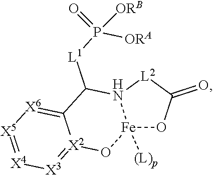

or a pharmaceutically acceptable salt thereof.

In some embodiments, the compound has formula:

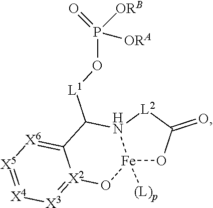

or a pharmaceutically acceptable salt thereof.

In some embodiments, the compound has formula:

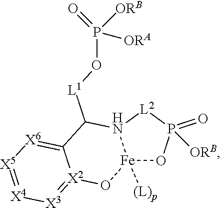

or a pharmaceutically acceptable salt thereof.

In some embodiments, the compound has formula:

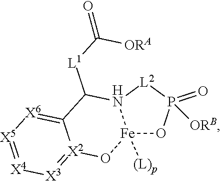

or a pharmaceutically acceptable salt thereof.

In some embodiments, the compound has formula:

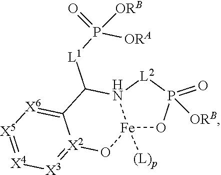

or a pharmaceutically acceptable salt thereof.

In some embodiments, the compound has formula:

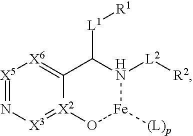

or a pharmaceutically acceptable salt thereof.

In some embodiments, the compound has formula:

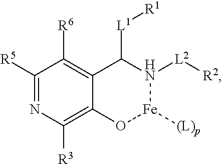

or a pharmaceutically acceptable salt thereof.

In some embodiments, the compound has formula:

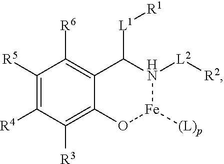

or a pharmaceutically acceptable salt thereof.

In some embodiments, at least one of R³, R⁴, R⁵, and R⁶ is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl), (C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸.

In some embodiments, at least one of R³, R^{3a}, R⁴, R^{4a}, R⁵, R^{5a}, R⁶, and R^{6a} is selected from C₁₋₃ alkyl, —(C₁₋₃ alkyl)_{q}OH, —(C₁₋₃ alkyl)_{q}SO₃R^{A}, —(C₁₋₃ alkyl)_{q}CO₂R^{A}, —(C₁₋₃ alkyl)_{q}OPO₃R^{A}R^{B}, and —(C₁₋₃ alkyl)_{q}PO₃R^{A}R⁸. In some embodiments, at least one of R³, R^{3a}, R⁴, R^{4a}, R⁵, R^{5a}, R⁶, and R^{6a} is selected from C₁₋₃ alkyl, CH₂OH, C(O)OH, SO₃H, and CH₂OP(O)(OH)₂.

In some embodiments, q is 0. In some embodiments, q is 1.

In some embodiments, each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.

In some embodiments, the compound is selected from any one of the following compounds:

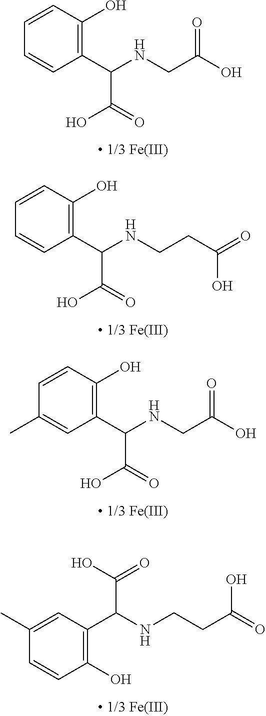 [SMILES: O=C(O)CNC(C(=O)O)c1ccccc1O ; O=C(O)CCNC(C(=O)O)c1ccccc1O ; Cc1ccc(O)c(C(NCC(=O)O)C(=O)O)c1 ; Cc1ccc(O)c(C(NCCC(=O)O)C(=O)O)c1]

or a pharmaceutically acceptable salt thereof.

### Pharmaceutically Acceptable Salts

In some embodiments, a salt of a compound of this disclosure (e.g., Formula A, (I), B, C, or D, or an iron complex thereof) is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the compounds of this disclosure, or pharmaceutically acceptable salts thereof, are substantially isolated.

### Methods of Use

In some embodiments, the present disclosure provides a method of treating iron deficiency anemia, the method comprising administering to a subject (e.g., in need thereof) a therapeutically effective amount of Fe³⁺ complex of a compound of Formula (I) as described herein, or a pharmaceutically acceptable salt thereof. In some embodiments, the method includes treating iron deficiency anemia, vitamin deficiency anemia, anemia of chronic disease, aplastic anemia, anemia associated with bone marrow disease, hemolytic anemia, sickle cell anemia, or thalassemia.

Some embodiments provide a method of increasing red blood cell production in a subject in need thereof, comprising administering to the subject a therapeutically effective amount of a compound described herein, or a pharmaceutically acceptable salt thereof.

Some embodiments provide a method of increasing hematocrit in a subject in need thereof, comprising administering to the subject a therapeutically effective amount of a compound described herein, or a pharmaceutically acceptable salt thereof.

Some embodiments provide a method of increasing blood iron levels in a subject in need thereof, comprising administering to the subject a therapeutically effective amount of a compound described herein, or a pharmaceutically acceptable salt thereof.

In some embodiments, the subject may be determined or identified as being in need of treatment by, e.g., a treating physician (for example, on the basis of diagnosis of anemia). Anemia may be diagnosed on the basis of laboratory tests as well as clinical presentation of one or more symptoms associated with anemia. In one example, anemia can be diagnosed on the basis of hematocrit (red blood cell count) less than 38% for a male subject and less than 35% for a female subject. In another example, anemia can be diagnosed on the basis of hemoglobin less than 11 g/dL for female subject and less than 12 g/dL for a male subject. Symptoms commonly associated with anemia include fatigue, weakness, pale skin, irregular heartbeats, shortness of breath, dizziness, lightheadedness, chest pain, a headache, or any combination of the foregoing. In yet another example, anemia may be diagnosed on the basis of identifying a mutation in a gene associated with anemia (e.g., a gene encoding hepcidin, ferroportin, or an associated biomolecule). In some embodiments, the method of treating of any of the aforementioned conditions includes a step of identifying a subject in need of treatment. In some embodiments, the method includes identifying a subject diagnosed with the anemia. In some embodiments, the method includes diagnosing the subject with anemia.

In some embodiments, administration of the iron complex compound of this disclosure results in alleviation of symptoms associated with anemia and leads to hematocrit and hemoglobin within the level of normal.

### Compositions, Formulations, and Routes of Administration

The present application also provides pharmaceutical compositions comprising an effective amount of a compound of the present disclosure (e.g., an iron complex of Formula (I), or a pharmaceutically acceptable salt thereof) disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, a compound of the present disclosure (e.g., an iron complex of a compound of Formula (I)) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound (e.g., an iron complex of a Formula (I)) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound such as an iron complex of Formula (I) is about 0.1 mg/kg, about 0.5 mg/kg, about 1 mg/kg, about 2 mg/kg, or about 5 mg/kg.

In some embodiments, an effective amount of the compound (e.g., an iron complex of a Formula (I)) can range, for example, from about 1 μg Fe/kg to about 500 μg Fe/kg, from about 2 μg Fe/kg to about 300 μg Fe, from about 5 μg Fe/kg to about 250 μg Fe/kg/kg, from about 1 μg Fe/kg to about 50 μg Fe/kg, from about 5 μg Fe/kg to about 100 μg Fe/kg/kg, from about 5 μg Fe/kg to about 50 μg Fe/kg/kg, from about 1 μg Fe/kg to about 25 μg Fe/kg/kg, or from about 1 μg Fe/kg to about 10 μg Fe/kg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein. In some embodiments, this disclosure provides a kit comprising (i) a container containing a compound of Formula (I), or a salt thereof; (ii); a container containing iron salt (e.g., water-soluble Fe³⁺ salt such as FeCl₃ or Fe₂(SO₄)₃; (iii) optionally a container containing pharmaceutically acceptable water or an aqueous solution (e.g., saline or dextrose solution); and (iv) optionally an insert containing guidelines for admixing the contents of container (i), the contents of container (ii), and pharmaceutically acceptable water or an aqueous solution (e.g., saline or dextrose solution), for example, the contents of the optional container (iii). The insert may also contain instructions and/or guidelines for administration of the admixed components to an anemia patient.

### Combinations

The compounds of the present disclosure can be used on combination with at least one medication or therapy useful, e.g., in treating or alleviating symptoms of iron deficiency anemia. Suitable examples of such medications include recombinant erythropoietin, vitamin B12, iron sucrose, nandrolone, ferrous fumarate, epoetin and derivatives thereof, triamcinolone, folic acid, leucovorin, procrin, ferralet, and the like. The iron complexes of this disclosure can also be co-administered with medications useful to treat or alleviate various co-morbidities. Suitable examples of such medications include anti-inflammatory agents (e.g., diclofenac, ibuprofen, indomethacin, ketoprofen, celecoxib, cortisone, prednisone, and the like). The compound of the present disclosure may be administered to the patient simultaneously with the additional therapeutic agent (in the same pharmaceutical composition or dosage form or in different compositions or dosage forms) or consecutively (the additional therapeutic agent may be administered in a separate pharmaceutical composition or dosage form before or after administration of the compound of the present disclosure).

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. In some embodiments, “alkylene” refers to a divalent alkyl group.

As used herein, the term “Cₙ₋ₘ haloalkyl”, employed alone or in combination with other terms, refers to an alkyl group having from one halogen atom to 2ₛ+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂. As used herein, the term “Cₙ₋ₘ alkylamino” refers to a group of formula —NH (alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms. Examples of alkylcarbonyl groups include, but are not limited to, methylcarbonyl, ethylcarbonyl, propylcarbonyl (e.g., n-propylcarbonyl and isopropylcarbonyl), butylcarbonyl (e.g., n-butylcarbonyl and tert-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂. As used herein, the term “Cₙ₋ₘ alkylaminosulfonyl” refers to a group of formula —S(O)₂NH (alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH (alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH (alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “carbamyl” to a group of formula —C(O)NH₂.

As used herein, the term “Cₙ₋ₘ alkylcarbamyl” refers to a group of formula —C(O)—NH (alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbonyl”, employed alone or in combination with other terms, refers to a —C(═O)— group, which may also be written as C(O).

As used herein, the term “carboxy” refers to a —C(O)OH group.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, “halo” refers to F, Cl, Br, or I. In some embodiments, a halo is F, Cl, or Br.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

The following examples are for illustrative purposes only and are not limiting.

### Example 1-N,N-bis(2-hydroxybenzyl)-L-glutamic acid (BBG)

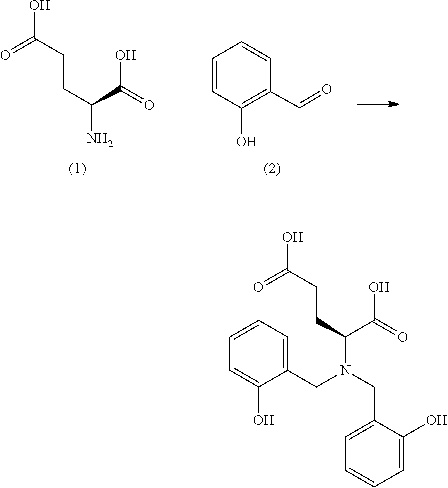 [SMILES: N[C@@H](CCC(=O)O)C(=O)O ; O=Cc1ccccc1O ; O=C(O)CC[C@@H](C(=O)O)N(Cc1ccccc1O)Cc1ccccc1O]

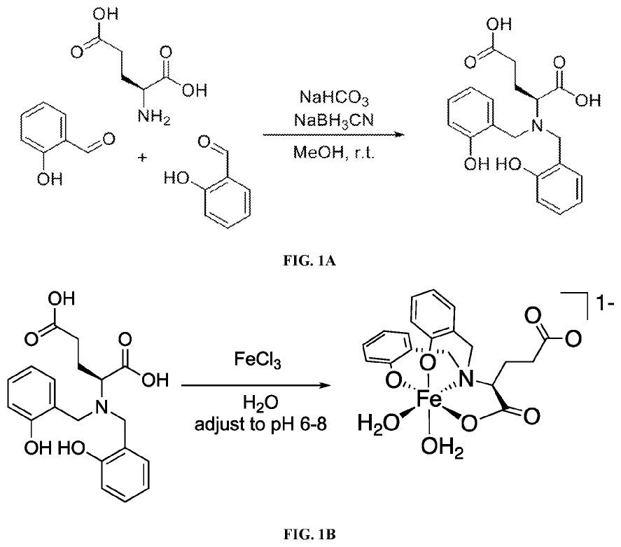

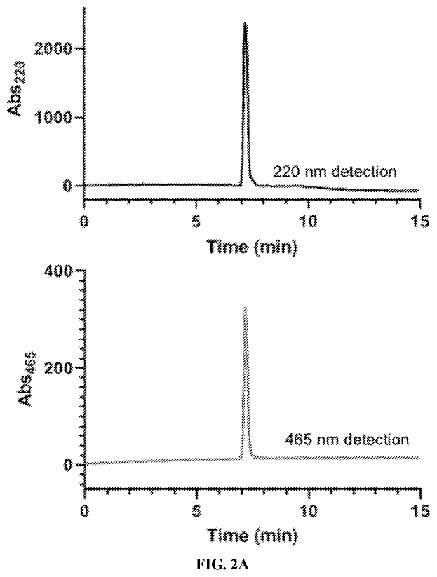

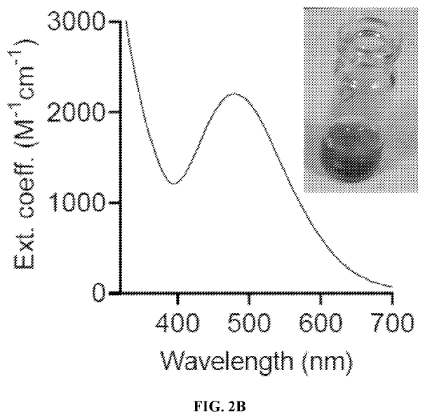

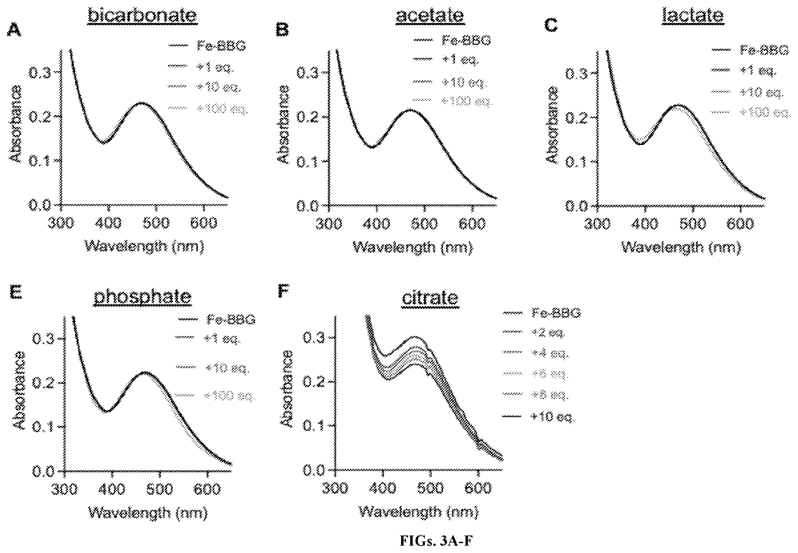

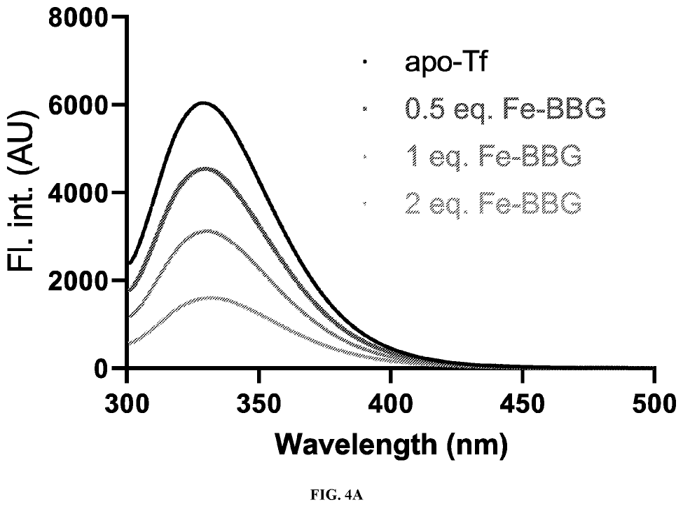

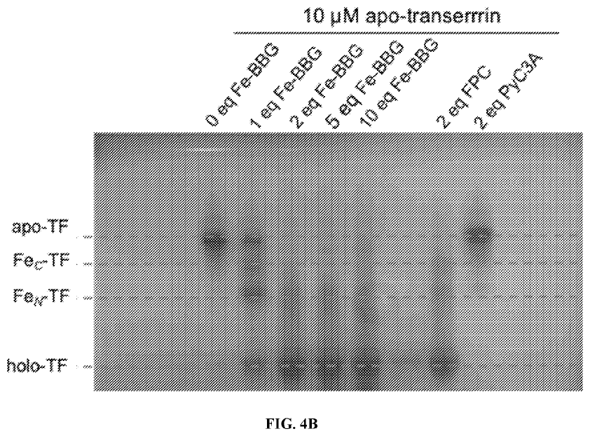

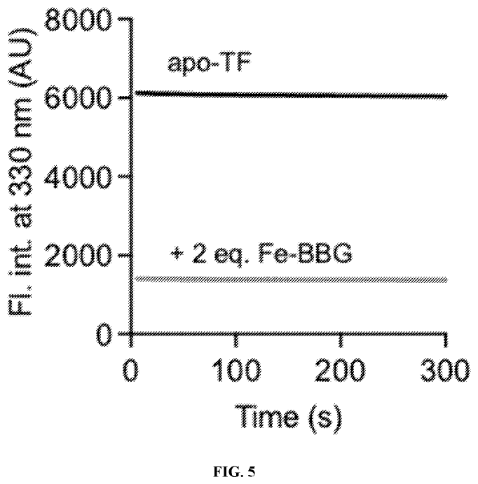

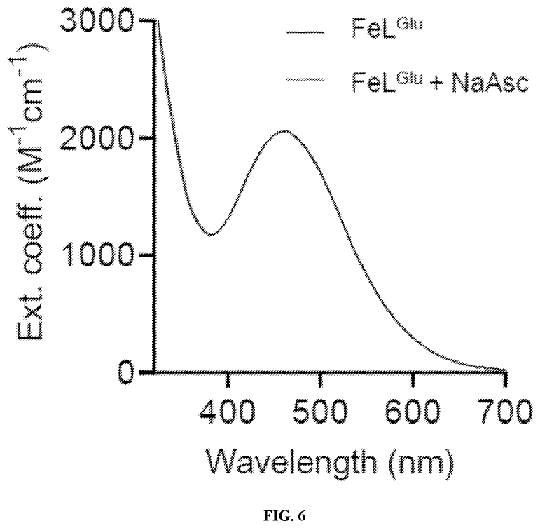

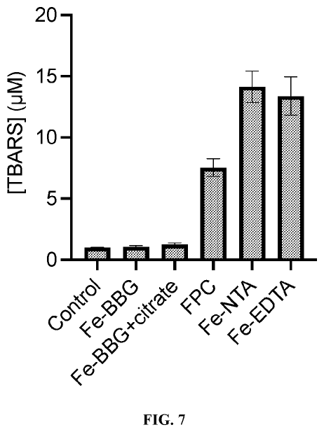

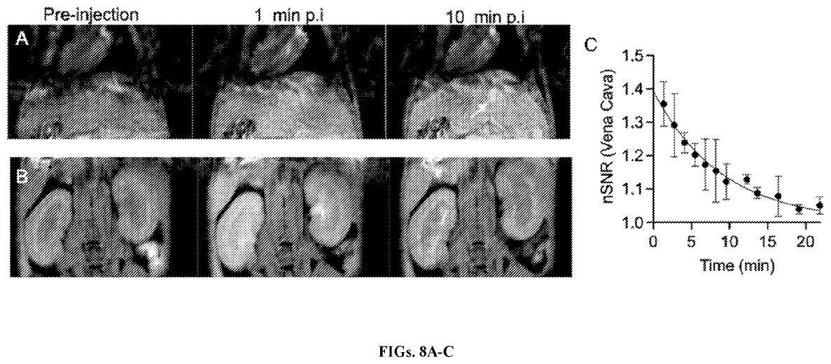

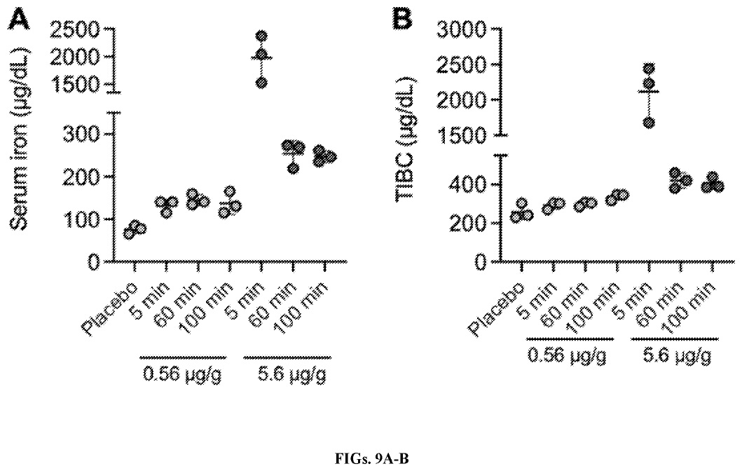

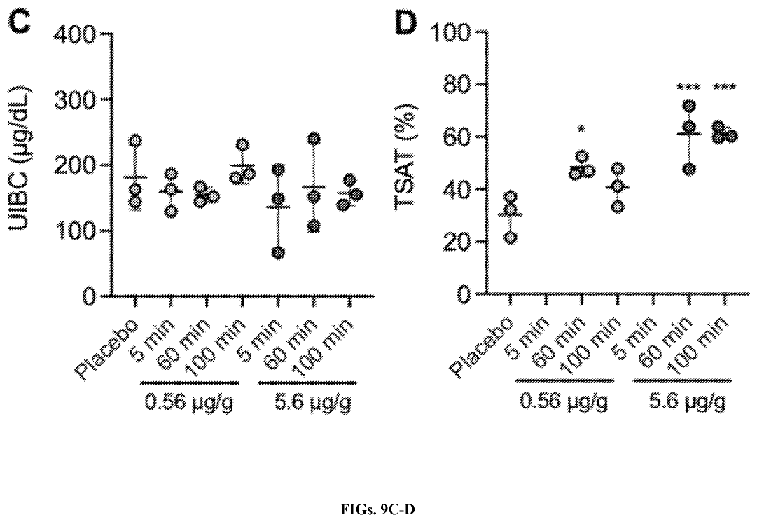

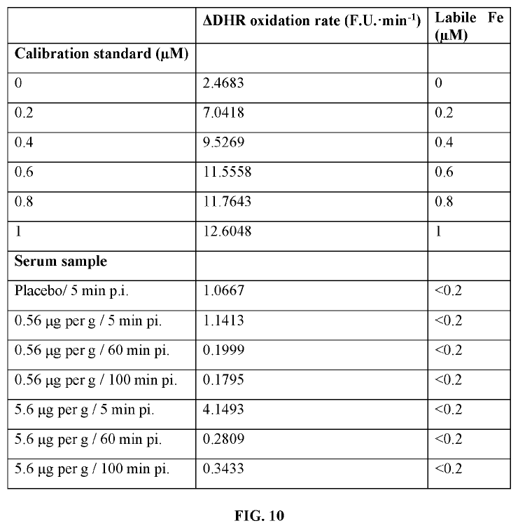

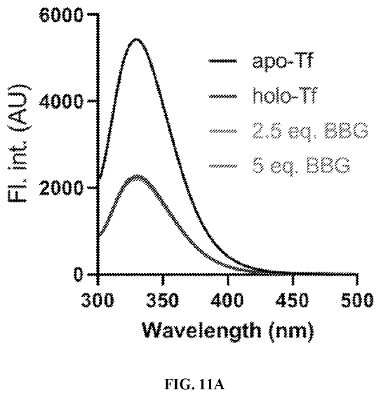

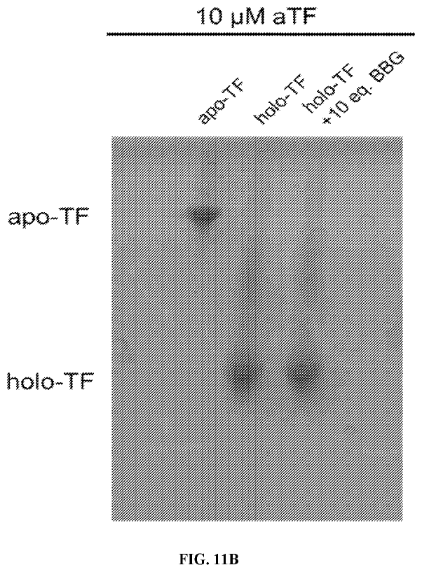

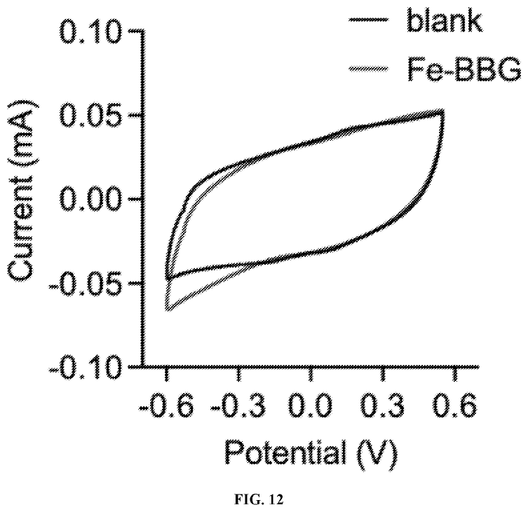

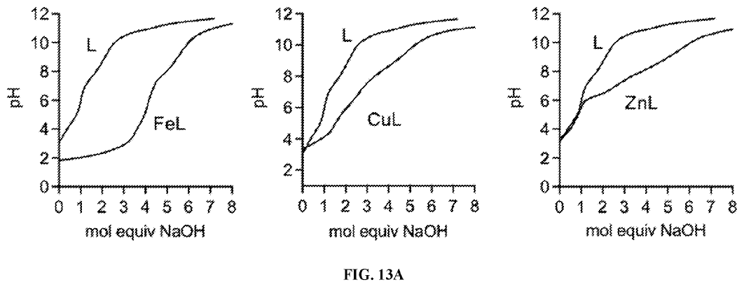

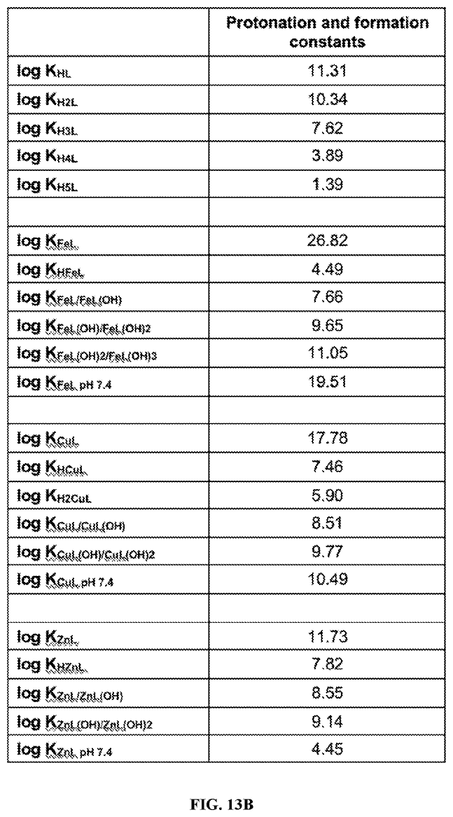

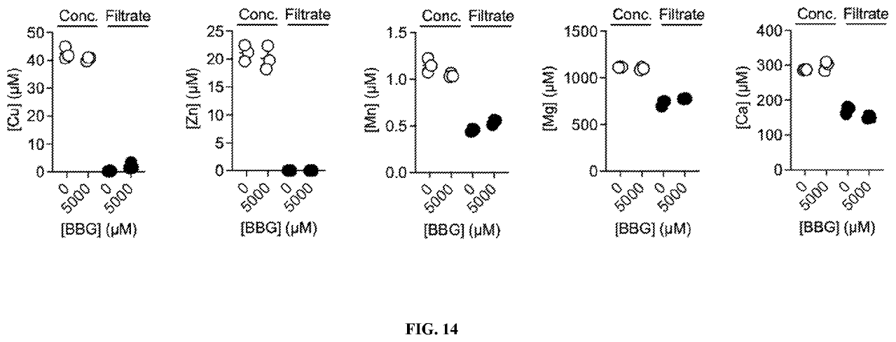

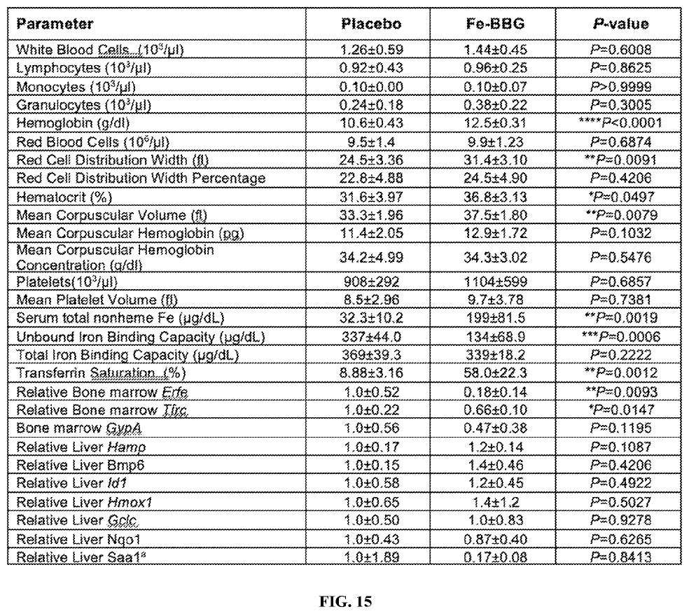

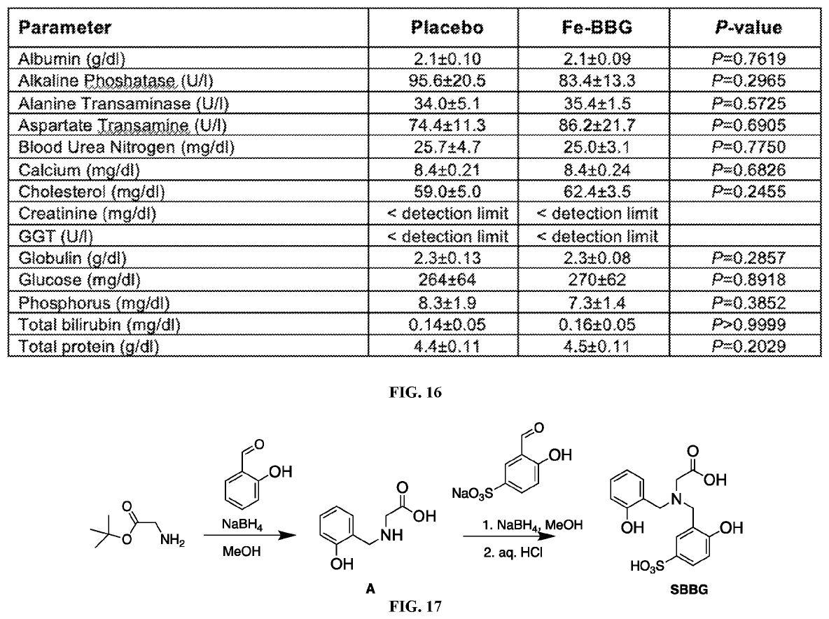

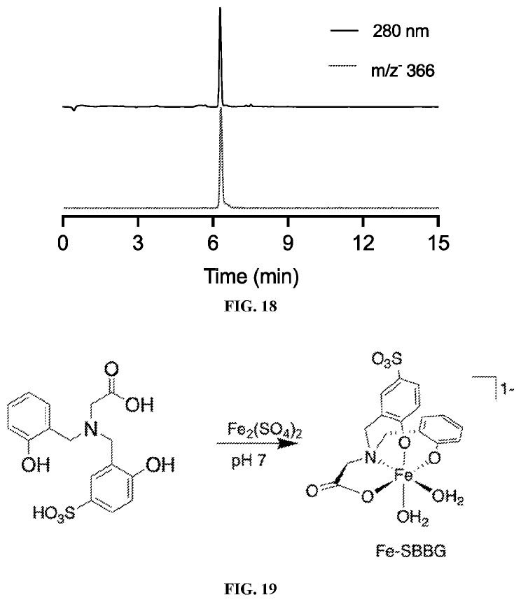

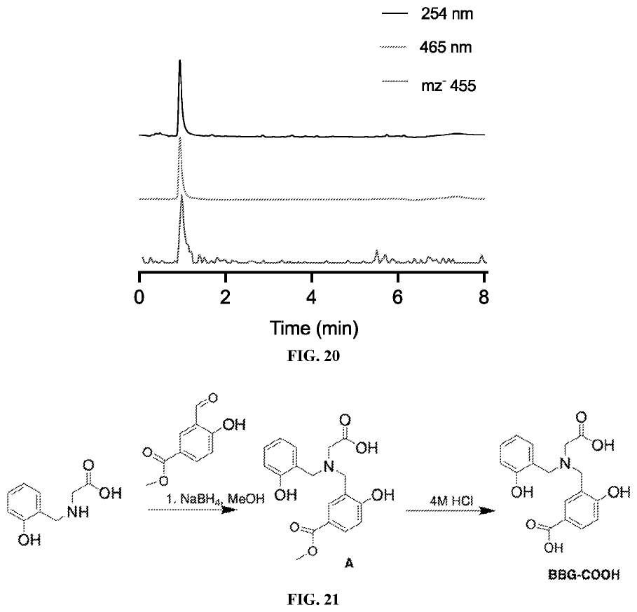

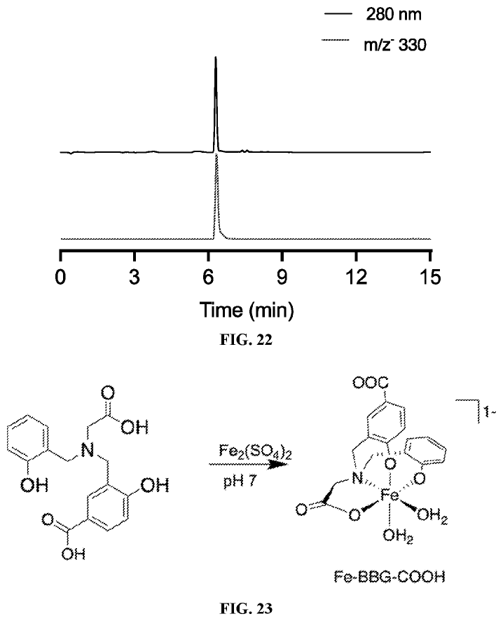

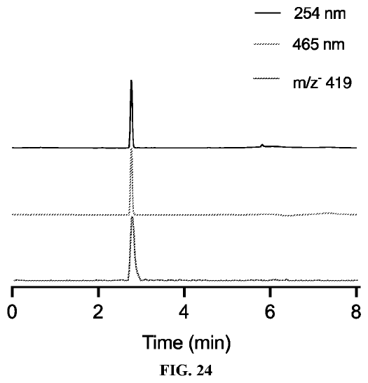

The reaction scheme is shown in FIG. 1A. L-glutamic acid (0.5 g, 3.4 mmol, 1 equiv.) and NaHCO₃ (2.3 g, 27.2 mmol, 8 equiv.) were mixed in 40 mL of MeOH. To the resulting suspension, salicylic aldehyde (1.7 g, 1.4 mL, 13.6 mmol, 4 equiv.) was added slowly under vigorous stirring. Then, the bright yellow reaction mixture was treated with sodium cyanoborohydride (1.1 g, 17 mmol, 5 equiv.) added in four portions of 0.275 g each over the course of 3 h. Vigorous stirring was continued at room temperature for a total of 3 h, after which the reaction mixture was filtered and the solvent removed to dryness by rotary evaporation. The crude product was re-dissolved in H₂O/MeCN (1:1, v/v) and purified by preparative RP-HPLC (Phenomenex Luna C5 column (250×21 20 mm); eluent A: H₂O/0.1% trifluoroacetic acid (TFA), eluent B: MeCN/0.1% TFA; gradient: 5-100% B over 30 min, flow rate 15 mL/min). The combined fractions were lyophilized to yield BBG TFA as an off-white hygroscopic powder (50 mg, 1·10⁻⁴ mol, 3%). UV-Vis: ε₂₈₀=7,884 M⁻¹ cm⁻¹. Elemental analysis calculated for C₁₉H₂₁NO₆·0.7 TFA·1 H₂O·0.025 MeCN: C, 53.61; H, 5.23; N, 3.13. Found: C, 53.58; H, 5.23; N, 3.13. QToF-MS for [C₁₉H₂₁NO₆+H]⁺: m/z [M+H]⁺=360.1447 (calcd.); m/z=360.1451 (exptl.). ¹H NMR (500 MHz, D₂O): δ, ppm 7.21-7.18 (m, 4H), 6.82-6.74 (m, 4H), 4.30 (s, 4H), 3.70 (m, 1H), 2.59-2.30 (m, 2H), 2.21 (m, 2H). ¹³C NMR (125.7 MHz, D₂O): 8, ppm 176.2, 170.4, 155.2, 132.1, 120.7, 116.2, 115.3, 63.0, 30.4, 20.3. ESI-MS for [C₁₉H₂₁NO₆+H]⁺: m/z [M+H]⁺=360.1 (calcd.); m/z=360.1 (exptl.).

### Example 2—Preparation of Fe-BBG

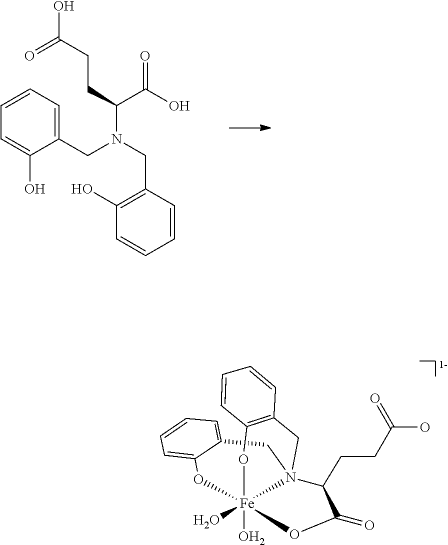

The reaction scheme is shown in FIG. 1B. BBG (9.5 mg, 2·10-5 mol, 1 equiv.) was dissolved in 4 mL of water. To the resulting solution, standardized solution of FeCl₃ (35.3 μL of 509.2 mM stock, 1.8.10-5 mol, 0.9 equiv.) was added dropwise under continuous stirring. The wine-red solution of the FeBBG complex was subsequently brought to pH 7.4 using 5 mM NaOH. The neutralized solution was desalted by preparative RP-HPLC HPLC (Phenomenex Luna C5 column (250×21 20 mm); eluent A: H₂O, eluent B: MeCN; gradient: 5-100% B over 30 min, flow rate 15 mL/min). The collected fractions were flash frozen in liquid nitrogen and lyophilized to give a dark red powder (7.9 mg, 1.7·10⁻⁵ mol, 93%). UV-Vis: ε₄₆₂=2,420 M⁻¹ cm⁻¹. QToF-MS for [C₁₉H₁₈FeNO₆+H]⁺: m/z [FeBBG+H]⁺=413.0578 (calcd.); m/z=413.0562 (exptl.) HPLC traces with 220 nm and 465 nm detection showing purity of isolate Fe-BBG is shown in FIG. 2A. The UV-vis spectrum of Fe-BBG is shown in FIG. 2B, with a photograph of a 0.1 mM solution of Fe-BBG in water shown in the inset.

### Example 3—In Vitro Assays to Evaluate Stability of Fe-BBG

FIG. 3 shows UV-vis traces of 0.1 mM Fe-BBG in the presence of between 0 and 100 molar equivalents of bicarbonate (panel A), acetate (panel B), lactate (panel C), phosphate (panel D), and between 0 and 10 molar equivalents citrate (panel E) in pH 7.4 buffered solution (50 mM HEPES buffer).

The UV-vis traces remain unchanged in the presence of up to 100 molar equivalents of bicarbonate, acetate, lactate, and phosphate. For solutions incubated with citrate, the equilibrium products composition was quantified based on absorbance at 465 nm, and from this data we estimate K_{comp}=0.006±0.002 for Fe-BBG challenge with citrate. In other words, the ligand BBG binds to Fe³⁺ with an estimated 230±40 times higher affinity than citrate.

The data demonstrate how Fe-BBG is stable against trans-chelation reactions using endogenously present chelators found often implicated in comprising speciation of non-transferrin bound iron.

### Example 4—In Vitro Assays to Evaluate Fe Delivery to Apo-Transferrin

Transfer of Fe from Fe-BBG to apo-transferrin can be monitored through quenching of the apo-transferrin intrinsic fluorescence. FIG. 4A shows Fluorescence spectra of 20 μM apo-transferrin after 2 h incubation with 0.5, 1, and 2 molar equivalents of FeBBG (50 mM HEPES buffer supplemented with 25 mM HCO₃⁻, pH 7.4; 37° C. λ_{Ex}=280 nm.).

Transfer of Fe from Fe-BBG to apo-transferrin can also be monitored using urea gel electrophoresis. FIG. 4B shows urea gel electrophoresis of apo-transferrin (apo-Tf), mono-ferric (FeC-TF and FeN-TF), and holo-transferrin (holo-Tf) from mixtures containing 10 μM transferrin incubated with 0, 1, 2, 5, and 10 molar equivalents Fe-BBG, or 2 molar equivalents Fe³⁺ as ferric pyrophosphate citrate (positive control), or 2 molar equivalents Fe³⁺ as the thermodynamically stable and kinetics inert complex Fe-PyC3A (negative control).

Together, the data in FIGS. 4A and 4B demonstrate how Fe-BBG is capable of stoichiometric transfer of Fe³⁺ to apo-transferrin.

### Example 5—Fe-BBG Rapidly Transfers Fe³⁺ to Apo-Transferrin In Vitro

FIG. 5 plots the time course of Fe³⁺ transfer of Fe-BBG to transferrin. The reaction was monitored through the quenching of apo-transferrin intrinsic fluorescence (conditions: 40 μM Fe-BBG, 20 μM apo-transferrin, 50 mM HEPES buffer supplemented with 25 mM HCO₃, pH 7.4; 25° C. λ_{Ex}=280 nm.). Fe³⁺ transfer from Fe-BBG to apo-transferrin was complete within 6 s. The temporal resolution of the instrument is 6 s, and the reaction is too fast for us to quantify kinetics.

### Example 6—Fe-BBG is not Reduced by Ascorbate

FIG. 6 shows UV-vis spectra of 0.1 mM Fe-BBG incubated in the absence or presence of 0.1 mM sodium ascorbate in 50 mM HEPES buffer, pH 7.4 The 462 nm absorption peak remains unchanged upon challenge with ascorbate.

### Example 7—Fe-BBG is Robust Against Reduction and Against Redox Cycling

To investigate whether Fe-BBG engages in spontaneous oxidation and reduction under forcing conditions, the compound was incubated with 5-deoxyribose for 1 h under previously described (Methods in Enzymology 1994, 233, 57-66) conditions designed to force production of deleterious hydroxyl radicals (5 mM Fe³⁺ complex, 2.8 mM H₂O₂, 2.8 mM deoxyribose, 0.1 mM ascorbic acid, 100 mM pH 7.4 phosphate buffer, 37° C.), and 5-deoxyribose oxidation products then quantified using thiobarbituric acid reactive substances (TBARS) assay. The complexes Fe-NTA and Fe-EDTA, both known to engage under redox cycling and to generate Fenton radicals under these reaction conditions, were incubated as positive controls. The reaction was run in the absence of any Fe as negative control. Ferric pyrophosphate citrate was also tested. FIG. 7 demonstrated that unlike the other Fe³⁺ complexes tested, incubation in the presence of Fe-BBG did not generate TBARS levels above those recorded in the absence of any Fe. Similarly, TBARS were not generated when Fe-BBG was reacted in the presence of physiologically relevant concentrations of citrate (iron citrate also engages in redox cycling), further underscoring the stability of Fe-BBG with respect to trans-chelation.

### Example 8—Pharmacokinetics of Fe-BBG Determined Using Dynamic Magnetic Resonance Imaging

Fe-BBG is paramagnetic, which enables us to monitor Fe-BBG blood clearance and excretion through dynamic magnetic resonance imaging. FIG. 8 shows coronal abdominal T₁-weighted images of a mouse prior to, 1 min, and 10 min after injection with Fe-BBG. Panel A shows liver enhancement. The gall bladder (yellow arrow) and bowels begin to contrast enhance at ˜10 min, consistent with partial hepatobiliary excretion The image series in panel B shows strong kidney enhancement 1 min after injection, which decreases rapidly and is strongly diminished by 10 min, consistent with rapid urinary excretion. Panel C shows Mono-exponential fits to plots of vena cava signal intensity vs. time indicate that blood MR signal decreases with a half-life of 5.5±2.8 min, consistent with rapid elimination.

### Example 9—In Vivo Assessment of Transferrin Saturation Increase

The in vivo efficacy of Fe-BBG as a direct-to-transferrin Fe replacement drug was preliminary evaluated in normal male C57BL/6mice. Mice received either 0.01 mmol Fe-BBG/kg (0.56 μg Fe per g body weight), 0.1 mmol Fe-BBG/kg (5.6 μg Fe per g body weight), or placebo (saline vehicle) via tail vein injection and were euthanized either 5 min, 60 min or 100 min post-injection. Serum nonheme iron parameters were then quantified using the spectrophotometric ferrozine assay.

FIG. 9 compares serum iron, total iron binding capacibity (TIBC), and unbound iron binding capacity (UIBC), recorded in mice treated with placebo and in serum harvested either 5 min, 60 min, or 100 min after Fe-BBG injection. The percentage transferrin saturation (% TSAT) was calculated at the 60 min and 100 min time points, at which point Fe-BBG and the BBG ligand remaining after trans-chelation with apo-transferrin had diminished to levels that will not interfere with the spectrophotometric assay.

The data demonstrate that Fe-BBG injection raises serum iron and TIBC, but does not decrease TIBC compared to treatment with placebo. The data also demonstrate how Fe-BBG elevates % TSAT significantly compared to placebo treated mice.

### Example 10—Fe-BBG does not Contribute Labile Iron In Vivo

To quantify labile iron released by Fe-BBG after intravenous injection, male C57BL/6 mice received either 0.01 mmol Fe-BBG/kg (0.56 μg Fe per g body weight), 0.1 mmol Fe-BBG/kg (5.6 μg Fe per g body weight), or placebo (saline vehicle) via tail vein injection and were euthanized either 5 min, 60 min or 100 min post-injection. Blood serum was harvested and labile Fe in each sample quantified by spectrophotometrically monitoring the rate of dihydrorhodamine 123 oxidation under redox forcing conditions, as reported in Blood 2003, 102 (7), 2670-2677 and described below:

Quadruplicates of 20 μL serum were transferred to clear-bottom 96-well plates. To 2 of the wells was added 180 μL iron-free HEPES buffered saline (HBS) (preincubated at 37° C.) containing 40 μM ascorbate and 50 M dihydrorhodamine 123 (DHR). To the other 2 wells was added 180 μL of the same solution containing 50 μM deferiprone. Immediately following reagent addition. The kinetics of fluorescence increase were followed at 37° C. in a Molecular Devices FlexStation 3 microplate reader with 485/538 nm excitation/emission filter pair, for 40 minutes, with readings every 2 minutes. The slopes (R) of DHR fluorescence intensity with time were calculated from measurements taken between 16 and 40 minutes and are given as FU·minute⁻¹ (fluorescence units per minute).

The duplicate values of R in the absence (R¹) and presence (R²) of deferiprone were averaged, and the LPI concentration (M) was determined from calibration curves relating the difference in slopes with and without deferiprone against iron concentration.

Calibration curves were obtained by spiking plasmalike medium (PLM) with Fe:NTA, 1:7 (mol:mol) to give final concentrations of 0.2 to 1 μM, followed by serial dilution in PLM and incubation for 30 minutes at 37° C. to allow binding of the Fe³⁺. Quadruplicates of 20 μL of these samples were assayed for LPI as described in the previous paragraphs. A standard curve of ADHR oxidation rates versus iron concentrations was built with data compiled from determinations done in PLM medium.

Plasma-like medium contained HEPES 20 mM, NaCl 150 mM, sodium citrate 120 μM, sodium ascorbate 40 μM, sodium phosphate dibasic 1.2 mM, sodium bicarbonate 10 mM, and human serum albumin 40 mg/mL (pH 7.4). Iron-free HEPES-buffered saline (HEPES 20 mM, NaCl 150 mM, pH 7.4) was obtained by treatment with 1 g chelex-100 per 100 mL solution.

The data shown in FIG. 10, indicate that Fe-BBG does not release labile iron in vivo, even in serum harvested 5 min after injection of 5.6 μg Fe/g as Fe-BBG, where total serum nonheme Fe is 25-fold higher than in placebo treated mice and well beyond native TBIC (see FIG. 9), DHR oxidation kinetics were consistent with concentrations <0.2 μM labile Fe.

In brief summary, the ligands of Formulae herein form iron complexes that are thermodynamically stable than transferrin, but are much more thermodynamically stable than the iron complexes formed with ligands such as carbonate, phosphate, acetate, lactate, and citrate, are not reduced by ascorbate, and do not engage in redox cycling with fenton radical generation under forcing conditions. The iron complexes formed by ligands of Formulae herein rapidly transfer Fe to transferrin in vitro, and also result in a significant increase in percentage transferrin saturation when injected to mice in vivo. Blood serum harvested from mice injection with the iron complexes formed by ligands of Formulae herein reveals no detectable labile iron. Taken together, this data supports these systems as effective and safe direct-to-transferrin iron replacement drugs.

### Example 11—the BBG Ligand does not Strip Fe from Transferrin

Solutions of 20 μM holo-transferrin and BBG of concentration ranging between 0 and 100 μM (0-5 molar equivalents) were incubated for 24 h in pH 7.4 50 mM HEPES buffer supplemented with 25 mM carbonate at 37° C., and Fe removal from transferrin was monitored using fluorescence spectroscopy (excitation wavelength: 280 nm). No evidence of Fe removal from transferrin was observed, FIG. 11A.

In another experiment, 10 M holo-transferrin and was incubated with 100 μM BBG (10 molar equivalents) for 24 h in pH 7.4 50 mM HEPES buffer supplemented with 25 mM carbonate at 37° C., and Fe removal monitored by urea gel electrophoresis assay, FIG. 11B. No evidence of Fe removal from transferrin was observed.

### Example 12—Cyclic Voltammetry of Fe-BBG

Cyclic voltammograms of pH 7.4 50 mM HEPES buffer solution the absence (black trace) and presence of 10 mM Fe-BBG (red trace). Glassy carbon working electrode, Pt counter electrode, electrolyte: 0.5M KNO₃, scan rate=300 mV/s, RT, FIG. 12.

### Example 13—the BBG Ligand Binds Fe³⁺ with High Specificity Over Other Metal Ions Present Endogenously In Vivo

The aqueous solutions thermodynamics of BBG and its corresponding complexes of Fe³⁺, Cu²⁺, and Zn²⁺ were interrogated through pH-potentiometric titration of BBG (L) in the absence and presence of 1 molar equivalent of Fe³⁺, Cu²⁺, or Zn²⁺, FIG. 13A.

pH-potentiometric measurements were performed using an Orion ROSS Ultra pH electrode and temperature-controlled reaction vessel held at 310 K. A standardized solution of 0.10 M NaOH was used as the titrant. The electrode was calibrated prior to each titration by titrating a standardized HCl(aq) solution at ionic strength 0.10 using NaCl as the inert electrolyte with the standardized NaOH titrant. A working slope and intercept were generated by plotting mV as a function of calculated pH, which enabled direct conversion of electrode readings to [H⁺] during sample titrations. pH values recorded during the titrations refers to hydrogen ion concentration. All titration samples were prepared in solutions of 0.10 M NaCl in distilled, deionized water. Ligand solutions were prepared by dissolving a weighed quantity into water and concentration was determined from the effective weight of the ligand and confirmed by the quantity of NaOH required to consume 1 molar equivalent of ligand proton. Solutions of 1:1 ligand: metal ion (Fe³⁺, Zn²⁺, Cu²⁺) were prepared by adding an appropriate volume of ICP-MS standardized metal ion solution to a weighed quantity of ligand, the solutions were then adjusted with water and 1 M NaCl to ionic strength I=0.1 M. The data was analyzed using the Hyperquad2013 software package. Specific experimental conditions are as follows: For BBG the starting titrand mixture contained 0.016 mmol L and 0.0872 mmol H⁺ in 2.7 mL, the titrant contained 0.1399 M NaOH. For 1:1 Fe: BBG, the starting titrand 0.0153 mmol Fe, 0.0153 mmol L, and 0.023 mmol H⁺ in 2.5 mL, the titrant contained 0.1399 M NaOH. For 1:1 Cu: BBG, the starting titrand mixture contained 0.0023 mmol Cu, 0.0023 mmol L, and 0.0230 mmol H⁺ in 2.6 mL, the titrant contained 0.1470 M NaOH. For 1:1 Zn: BBG, the starting titrand 0.0025 mmol Zn, 0.0025 mmol L, and 0.024 mmol HT in 2.6 mL, the titrant contained 0.1470 M NaOH.

The entirety of the thermodynamics parameters are tabulated in FIG. 13B. The conditional pH 7.4 stability constant of Fe-BBG (log K_{FeL PH 7.4}) is 19.51, whearas fo Cu-BBG and Zn-BBG, the log K_{CuL PH 7.4} and log K_{ZnL PH 7.4} values are 10.49 and 4.49, respectively. BBG binds Fe³⁺ with 9-orders of magnitude greater stability than Cu²⁺, and 15 orders of magnitude greater stability than Zn²⁺.

### Example 14—BBG does not Compete Strongly for Metals Endogenously Present in Blood Serum

Based on the log K_{ML pH 7.4} values for Zn²⁺-BBG and Cu²⁺-BBG (4.45 and 10.49, respectively), we do not expect the BBG ligand to sequester or redistribute metal ions present in blood serum and the extracellular spaces. For example, serum Zn²⁺ speciation is comprised predominantly of Zn-bound to albumin with log K_{ML PH 7.4}=7.0 (Biochim Biophys Acta 2013, 1830 (12), 5444-5455), whereas serum Cu speciation comprises Cu²⁺ bound to proteins including albumin, a-macroglobulin, and low molecular weight ligands, each with log K_{ML PH 74}˜13 (Coord. Chem. Rev. 2021, 433, 213727). Given the Cu-BBG and Zn-BBG stability constants and high abundance of serum albumin (˜660 μM) we posit that even millimolar concentrations of BBG should not disrupt homeostasis of these metal ions.

To illustrate this point, human blood plasma samples containing between 0-5 mM BBG were incubated for 1 h at 37° C. before separation of the low-molecular weight solution components by ultrafiltration through a 10 kDa molecular weight cutoff filter. The plasma concentrate and ultrafiltrate were assayed for concentrations of Cu and Zn, as well as Mn, Mg, and Ca by ICP-MS, FIG. 14. Incubation with BBG had little effect on the metal ion content remaining in the plasma concentrate, nor did BBG substantially increase concentrations of any metal ion in the ultrafiltrate relative to control samples not treated with any ligand.

### Example 15—Treatment with Fe-BBG Corrects Anemia in Mouse Model of Iron Refractory Iron Deficiency Anemia

We evaluated the therapeutic efficacy of Fe-BBG in Tmprss6 knockout mice, which exhibit aberrantly high levels of hepcidin and suffer Fe restricted erythropoiesis, phenocopying human patients with IRIDA due to TMPRSS6 mutations, and recapitulating salient features of anemia of inflammation. Tmprss6 knockout mice received 29 intraperitoneal injections each containing 25 μg Fe as Fe-BBG (1.3±0.18 μg Fe per gram body weight, N=5 mice, 2 male, 3 female) or containing saline (placebo, N=5 mice, 2 male, 3 female) over 15 days. The cumulative dose of Fe was 725 μg Fe (37±0.53 μg Fe per gram body weight). The mice were euthanized 120 min after the final injection. Blood was harvested for measurement of complete blood counts, and nonheme Fe parameters. Bone marrow and liver tissue were analyzed by qPCR for expression of genes related to erythropoietic activity, Fe exposure, oxidative stress, and inflammation, the data are tabulated in FIG. 15. Clinical chemistry was also recorded, see Example 16 below. FIG. 15 data are shown as mean±standard deviation. Statistical differences between groups were determined by two-tailed Student's t-test for normally distributed values or Mann-Whitney (/test for non-normally distributed values. Significant differences are indicated by *P<0.05, **P<0.01, ****P<0.0001.

Treatment with Fe-BBG resulted in a significant elevation of hemoglobin compared to placebo. Hematocrit levels were also increased significantly. Fe-BBG treatment significantly increased mean corpuscle volume and red cell width, which we attribute to newly increased production of larger hemoglobin replete erythrocytes. Taken together, these changes are consistent with restoration of normal hematology. No noteworthy differences were detected between any other hematology parameters.

Serum Fe levels were elevated in Fe-BBG treated mice. which was mirrored by concomitant decrease in unbound iron binding capacity (UIBC). Total iron binding capacity (TIBC) levels in Fe-BBG treated mice were unchanged relative to placebo, indicating that Fe-BBG has cleared entirely 120 min after the final injection and that serum Fe increase arises from replenishment of the transferrin Fe pool. In this regard, percentage transferrin saturation increase (% TSAT) is increased significantly in Fe-BBG treated mice, with mean % TSAT increased by nearly 7-fold.

The qPCR data data demonstrate that bone marrow erythroferrone (Erfe, 5J) is significantly downregulated in Fe-BBG treated mice, consistent with re-hemoglobinization. Transferrin receptor type 1 (Tfrc, 5K) and glycophorin A (GypA), proteins abundantly expressed by erythroid precursor cells, were also downregulated in Fe-BBG treated mice, consistent with restoration of normative red blood cell maturation processes.

### Example 16—Fe-BBG is Well Tolerated by Mice after Repeat Dosing

Repeat dosing of Fe-BBG to Tmprss6 knockout mice did not generate significant differences in any clinical chemistry parameters, including serum markers routinely used in drug toxicity screens, FIG. 16. FIG. 16 data are shown as mean±standard deviation. Statistical differences between groups were determined by two-tailed Student's t-test for normally distributed values or Mann-Whitney U test for non-normally distributed values. Significant differences are indicated by *P<0.05, **P<0.01, ***P<0.001, ****P<0.0001.

Fe-BBG did not trigger upregulation of liver heme oxygenase 1 (Hmox1), glutamate-cysteine ligase catalytic subunit (Gclc), or NAD (P) H quinone dehydrogenase 1 (NqoI), which taken together indicate that Fe-BBG did not result in oxidative stress. Expression of Serum amyloid A1 (SaaI), a sensitive marker of inflammation, was also unchanged after repeat dosing with Fe-BBG, FIG. 15.

### Example 17—N-(2-hydroxy-5-sulfobenzyl)-N-(2-hydroxybenzyl)glycine (SBBG)

The reaction scheme is shown in FIG. 17.

Tert-butyl (2-hydroxybenzyl)glycinate (A). In 50 mL methanol, tert-butyl glycinate (0.69 g, 4.1 mmol, 1 equiv.) and sodium bicarbonate (1.38 g, 16.4 mmol, 4 equiv.) were mixed for several minutes. Salicylaldehyde (0.5 g, 4.1 mmol, 1 equiv.) was added slowly while stirring. After 15 minutes, sodium borohydride (0.16 g, 4.1 mmol, 1 equiv.) was added in four portions over two hours. The white mixture was then stirred for an additional two hours, after which it was filtered, and the solvent removed by rotary evaporation. The crude product was dissolved in ethyl acetate and washed with saturated aqueous sodium bicarbonate and dried in vacuo, yielding a white powder (0.18 g, 0.7 mmol, 18%). ¹H NMR (500 MHz, CD₃OD): δ, ppm 6.97-7.09 (m, 2H), 6.65-6.78 (m, 2H), 3.77 (s, 2H), 3.21 (s, 2H), 1.41 (s, 9H). ¹³C NMR (125.7 MHz, CD₃OD): δ, ppm 171.55, 157.09, 129.92, 128.98, 124.36, 119.57, 115.54, 81.59, 53.79, 52.13, 45.81, 44.15, 27.47.

### N-(2-hydroxy-5-sulfobenzyl)-N-(2-hydroxybenzyl)glycine

In 10 mL methanol, Intermediate A (0.1 g, 0.42 mmol, 1 equiv.) and sodium bicarbonate (0.13 g, 1.6 mmol, 4 equiv.) were combined. Monosodium 5-sulfonatosalicylaldehyde (prepared as described in Dalton Trans., 2012, 41, 13927-13935) (0.09 g, 0.4 mmol, 1 equiv.) was added to the mixture slowly while stirring. Sodium borohydride (0.016 g, 0.4 mmol, 1 equiv.) was added in four portions over two hours, and the mixture was then stirred for an additional two hours. The reaction mixture was filtered, and the solvent removed by rotary evaporation. The crude product was dissolved in water and washed with ethyl ether. The product was dried in vacuo and deprotected by stirring in 6 M hydrochloric acid (5 mL) for two hours. SBBG was purified by preparative scale RP-HPLC using a Phenomenex Luna C5 column (250×21 20 mm); eluent A: H2O, eluent B: MeCN; gradient: 5-100% B over 30 min; flow rate 15 mL/min. The fractions were analyzed using LC/MS and fractions containing pure product were combined, frozen with liquid nitrogen, and lyophilized to dryness to yield a beige powder (0.1640 g, 0.39 mmol, 92%). ¹H NMR (500 MHz, D₂O): δ, ppm 7.53-7.64 (m, 1H), 7.36-7.41 (m, 1H), 7.24-7.29 (m, 1H), 7.00-7.13 (1H), 6.48-6.63 (m, 3H), 3.65-3.77 (m, 4H), 3.16 (s, 2H). ESI-MS for [C₁₆H₁₆NO₇S]⁻: m/z [M−H]⁺=366.1 (calcd.); m/z=366.1 (exptl.). LC-MS characterization of SBBG is shown in FIG. 18.

### Example 18—Preparation of Fe-SBBG

The reaction scheme is shown in FIG. 19. A batch of SBBG (15 mg, 0.041 mmol) in 4 mL water was mixed with Fez (SO₄) 3 (8 mg, 0.040 mmol of Fe). The solution turned a deep-red color and was adjusted to pH 7.40 using 12.5 mM NaOH (aq). Fe-SBBG was then purified by preparative scale RP-HPLC using a Phenomenex Luna C18 column (250×21 20 mm); eluent A: H₂O, eluent B: MeCN; gradient: 5-95% B over 30 min; flow rate 15 mL/min. The fractions were analyzed using LC/MS and the fractions containing pure product were combined, frozen with liquid nitrogen, and lyophilized to dryness to yield a dark red powder (15 mg, 0.034 mmol, 84%). ESI-MS for [C₁₆H₁₇FeNO₉S]⁻: m/z [M−H+2H₂O]⁻=455.0 (calcd.); m/z=455.0 (exptl.). LC-MS characterization of SBBG is shown in FIG. 20

### Example 19—3-(((carboxymethyl)(2-hydroxybenzyl)amino)methyl))-4-hydroxybenzoic acid (BBG-COOH)

The reaction scheme is shown in FIG. 21.

### N-(2-hydroxy-5-(methoxycarbonyl)benzyl)-N-(2-hydroxybenzyl)glycine) (A)

A batch of N-(2-hydroxymethyl)glycine (0.774 g, 4.27 mmol) was stirred with sodium bicarbonate (2.17 g, 25.8 mmol) in 25 mL methanol. To this mixture was added methyl 3-formyl-4-hydroxybenzoate (1.08 g, 5.99 mmol, added in 4 equal portions) and sodium borohydride (110 mg, 2.91 mmol, added in 4 equal portions, each portion added after 3-formyl-4-hydroxybenzoate) and the reaction monitored using HPLC. The reaction was completed when the relative peak area of starting material to intermediate A was roughly 1:4 using 280 nm detection. The reaction was then concentrated to dryness, partitioned between 50 mL H₂O and 50 mL EtOAc, separated, and then the aqueous portion washed 2× more with 50 mL ethyl acetate. EtOAc layer was then adjusted to pH 6-7 using 1M HCl/1M NaOH and concentrated to a residue. The concentrated residue was taken up in 10 mL 1:1 H₂O:MeOH, and purified by preparative RP-HPLC (Teledyne ISCO, RediSep C₁₈ Gold column (150 g); eluent A: H₂O adjusted to pH 3.0, eluent B: MeCN; gradient: 5-50% B over 6 column volumes, flow rate 85 mL/min). The fractions were analyzed using LC/MS and fractions containing product were combined, frozen with liquid nitrogen, and lyophilized to dryness to yield a white powder (0.42 g, the isolated material contained both Intermediate A and 4-hydroxy-3-(hydroxymethyl)benzoate by-product) ESI-MS for [C₁₈H₁₉NO₆+H]⁺: m/z [M+H]⁺=346.1 (calcd.); m/z=356.1 (exptl.).

### 3-(((carboxymethyl)(2-hydroxybenzyl)amino)methyl))-4-hydroxybenzoic acid

The solids containing intermediate A (0.42 g) were stirred in 20 mL 4M HCl (aq) and heated to 90° C. for 2 hours. The mixture was then concentrated to dryness and triturated with 25 mL H₂O. The resultant solids wer taken up in MeOH and purified by preparative RP-HPLC (Teledyne ISCO, RediSep C18 Gold column (150 g); eluent A: H₂O adjusted to pH 3.0, eluent B: MeCN; gradient: 5-50% B over 6 column volumes, flow rate 85 mL/min). The fractions were analyzed using LC/MS and fractions containing product were combined, frozen with liquid nitrogen, and lyophilized to dryness to yield a white powder (0.075 g, 0.227 mmol, 5% overall yield starting from N-(2-hydroxybenzyl)glycine). ¹H NMR (500 MHz, do-DMSO): δ, ppm 7.78 (s, 1H), 7.73 (d, 1H), 7.11 (m 2H), 6.85 (d, 1H), 6.77 (m, 2H), 3.83 (s, 2H), 3.77 (s, 2H), 3.23 (s, 2H). ¹³C NMR (500 MHz, d₆-DMSO): δ, ppm 172.1, 167.2, 160.8, 156.5, 132.5, 131.2, 130.8, 129.3, 123.1, 122.9, 121.9, 119.5, 115.9, 115.8, 53.7, 53.6, 53.5. ESI-MS for [C₁₇H₁₆NO₆]: m/z [M−H]⁻=330.1 (calcd.); m/z=330.1 (exptl.). LC-MS characterization of BBG-COOH is shown in FIG. 22.

### Example 20—Preparation of Fe-BBG-COOH

The reaction scheme is shown in FIG. 23. A batch of BBG-COOH (17 mg, 0.051 mmol) was mixed in 3 mL water and 12.5 mM NaOH (aq) was added dropwise until the solids dissolved. To this mixture was added Fez (SO₄) 3 (10 mg, 0.050 mmol of Fe). The mixture turned a deep-red color and was heterogenous. The mixture and was adjusted to pH 7.10 using 12.5 mM NaOH (aq), and the solution filtered yielding a dark red homogenous filtrate. Fe-BBG-COOH was then purified by preparative scale RP-HPLC using a Phenomenex Luna C18 column (250×21 20 mm); eluent A: H₂O, eluent B: MeCN; gradient: 5-95% B over 30 min; flow rate 15 mL/min. The fractions were analyzed using LC/MS and the fractions containing pure product were combined, frozen with liquid nitrogen, and lyophilized to dryness to yield a dark red powder (7.0 g, XX mmol, 68%). ESI-MS for [C₁₇H₁₇FeNO₈]⁻: m/z [M−H+2H₂O]⁻=419.0 (calcd.); m/z=419.0 (exptl.). LC-MS characterization of SBBG is shown in FIG. 24

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A compound which is a Fe³⁺ complex of Formula (A-I): 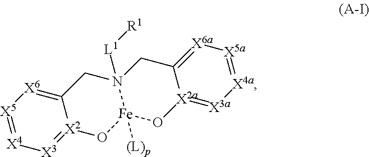
or a pharmaceutically acceptable salt thereof, wherein: [symbol]
indicates a coordinate bond;
each L is independently a Fe³⁺ ligand;
p is 0, 1 or 2;
L¹ is —C₁₋₃ alkylene-, optionally substituted with R¹⁰;
each R¹⁰ is independently —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, or —(C₁₋₃ alkyl), (C═O)NHSO₂R⁸;
R¹ is selected from C(O)O, P(O)(O)(OR^{B}), and (C═O)NR¹⁰;
each q is independently 0 or 1;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X³, and X⁶ are N;
X^{2a} is selected from N and C;
X^{3a} is selected from N and CR^{3a};
X⁴a is selected from N and CR^{4a};
X^{5a} is selected from N and CR^{5a},
X^{6a} is selected from N and CR^{6a};
provided that no more than two of X^{2a}, X^{3a}, X^{4a}, X^{5a}, and X^{6a} are N;
R³, R^{3a}, R⁴, R^{4a}, R⁵, R^{5a}, R⁶, and R^{6a} are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN;
provided that if L¹-R¹ is CH₂C(O)O or CH₂CH₂C(O)O, then R⁵ and R^{5a} are not both S(O)₂OH, methyl, halo, or tert-butyl.

2. The compound of claim 1, wherein the compound has formula: 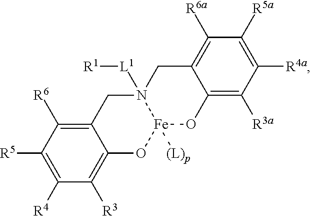
or a pharmaceutically acceptable salt thereof.

3. The compound of claim 1, wherein the compound has formula: 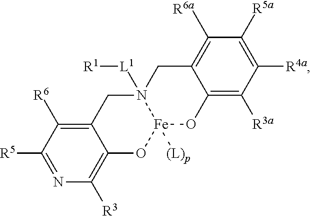
or a pharmaceutically acceptable salt thereof.

4. The compound of claim 1, wherein the compound has formula: 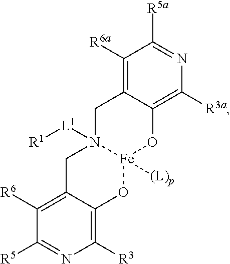
or a pharmaceutically acceptable salt thereof.

5. The compound of any one of claims 1-4, wherein L¹ is —C₁₋₃ alkylene-.

6. The compound of any one of claims 1-4, wherein L¹ is —C₁₋₃ alkylene-substituted with R¹⁰.

7. The compound of any one of claims 1-4, wherein L¹ is selected from methylene, ethylene, and propylene.

8. The compound of any one of claims 1-7, wherein the compound has formula: 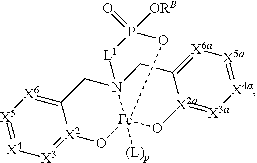
or a pharmaceutically acceptable salt thereof.

9. The compound of any one of claims 1-7, wherein the compound has formula: 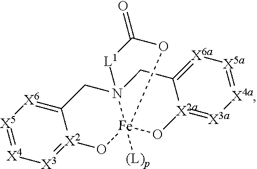
or a pharmaceutically acceptable salt thereof.

10. The compound of claim 9, having formula: 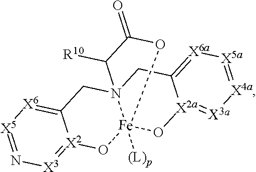
or a pharmaceutically acceptable salt thereof.

11. The compound of claim 9, having formula: 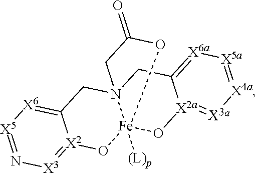
or a pharmaceutically acceptable salt thereof.

12. The compound of claim 11, having formula: 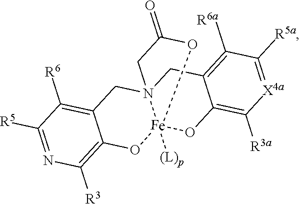
or a pharmaceutically acceptable salt thereof.

13. The compound of claim 9, selected from: 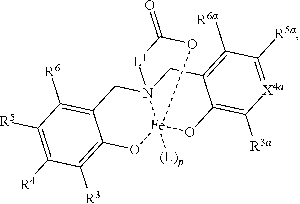
or a pharmaceutically acceptable salt thereof, wherein:
L¹ is selected from methylene and ethylene,
R^{5a} is H; and
R⁵ is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰.

14. The compound of any one of claims 1-13, wherein at least one of R³, R^{3a}, R⁴, R^{4a}, R⁵, R^{5a}, R⁶, and R^{6a} is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰.

15. The compound of claim 14, wherein at least one of R³, R^{3a}, R⁴, R^{4a}, R⁵, R^{5a}, R⁶, and R^{6a} is R¹⁰.

16. The compound of any one of claims 1-15, wherein R¹⁰ is selected from —(C₁₋₃ alkyl)_{q}CO₂R^{A}, —(C₁₋₃ alkyl)_{q}OP(R⁸)O₂R^{B}, —(C₁₋₃ alkyl)_{q}(C═O)NHSO₂R⁸, and —(C₁₋₃ alkyl)_{q}PO₃R^{A}R⁸.

17. The compound of any one of claims 1-15, wherein R¹⁰ is —(C₁₋₃ alkyl)_{q}CO₂R^{A}.

18. The compound of any one of claims 1-17, wherein q is 0.

19. The compound of any one of claims 1-17, wherein q is 1.

20. The compound of any one of claims 1-19, wherein each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.

21. The compound of claim 1, wherein the compound has formula: 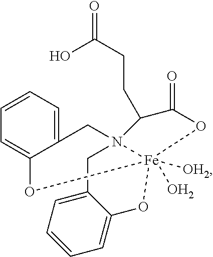
or a pharmaceutically acceptable salt thereof.

22. A compound which is a Fe³⁺ complex of Formula (B-I): 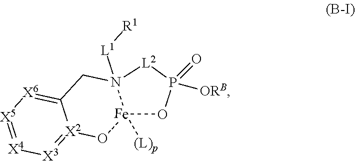
or a pharmaceutically acceptable salt thereof, wherein: [symbol]
indicates a coordinate bond;
each L is independently a Fe³⁺ ligand;
p is 0, 1 or 2;
L¹ is —C₁₋₃ alkylene-, optionally substituted with R¹⁰;
L² is —C₁₋₃ alkylene-, optionally substituted with R¹⁰;
each R¹⁰ is independently —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl), (C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, or —(C₁₋₃ alkyl), (C═O)NHSO₂R⁸;
R¹ is selected from CO₂R^{A}, P(O)(OR^{A})(OR^{B}), and (C═O)NR^{A}R¹⁰;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
R³, R⁴, R⁵, and R⁶ and are each independently selected from H, C₁₋₃ alkyl, C₁₋₃ alkoxy, C_{1.3} haloalkyl, C₁₋₃ haloalkoxy, halo, —(C₁₋₃ alkyl)_{q}OH, —(C₁₋₃ alkyl)_{q}CN, and R¹⁰;
each R⁸ is selected from C_{3.9} alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN.

23. The compound of claim 22, wherein L² is —C₁₋₃ alkylene-.

24. The compound of claim 22, wherein L² is —C₁₋₃ alkylene-substituted with R¹⁰.

25. The compound of claim 22, wherein L² is selected from methylene and ethylene.

26. The compound of any one of claims 22-25, wherein L¹ is —C₁₋₃ alkylene-.

27. The compound of any one of claims 22-25, wherein L¹ is —C₁₋₃ alkylene-substituted with R¹⁰.

28. The compound of any one of claims 22-25, wherein L¹ is selected from methylene and ethylene.

29. The compound of any one of claims 22-28, wherein R¹ is (C═O)NR^{A}R¹⁰.

30. The compound of any one of claims 22-28, wherein the compound has formula: 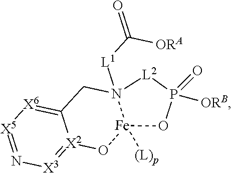
or pharmaceutically acceptable salt thereof.

31. The compound of any one of claims 22-28, wherein the compound has formula: 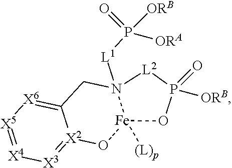
or pharmaceutically acceptable salt thereof.

32. The compound of any one of claims 22-28, wherein the compound has formula: 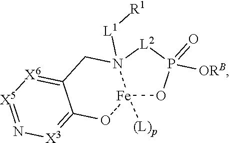
or pharmaceutically acceptable salt thereof.

33. The compound of any one of claims 22-28, wherein the compound has formula: 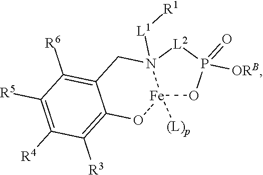
or pharmaceutically acceptable salt thereof.

34. The compound of any one of claims 22-33, wherein at least one of R³, R⁴, R⁵, and R⁶ is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰.

35. The compound of any one of claims 22-33, wherein at least one of R³, R⁴, R⁵, and R⁶ is R¹⁰.

36. The compound of any one of claims 22-35, wherein R¹⁰ is selected from —(C₁₋₃ alkyl)_{q}CO₂R^{A}, —(C₁₋₃ alkyl)_{q}OP(R⁸)O₂R^{B}, —(C₁₋₃ alkyl), (C═O)NHSO₂R⁸, and —(C₁₋₃ alkyl)_{q}PO₃R^{A}R⁸.

37. The compound of claim 36, wherein R¹⁰ is —(C₁₋₃ alkyl)_{q}CO₂R^{A}.

38. The compound of any one of claims 22-37, wherein q is 0.

39. The compound of any one of claims 22-37, wherein q is 1.

40. The compound of any one of claims 22-39, wherein each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.

41. A compound which is a Fe³⁺ complex of Formula (C-I): 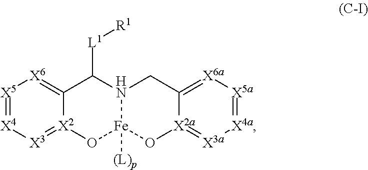
or a pharmaceutically acceptable salt thereof, wherein: [symbol]
indicates a coordinate bond;
each L is independently a Fe³⁺ ligand;
p is 0, 1 or 2;
L¹ is —C₁₋₃ alkylene-, or L¹ is absent;
R¹ is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
X^{2a} is selected from N and C;
X^{3a} is selected from N and CR^{3a};
X⁴a is selected from N and CR^{4a};
X^{5a} is selected from N and CR^{5a};
X^{6a} is selected from N and CR⁶a;
provided that no more than two of X^{2a}, X^{3a}, X^{4a}, X^{5a}, and X^{6a} are N;
R³, R^{3a}, R⁴, R^{4a}, R⁵, R^{5a}, R⁶, and R^{6a} are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl), (C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸;
each q is independently 0 or 1;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁-3 haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN.

42. The compound of claim 41, wherein L¹ is absent.

43. The compound of claim 41, wherein L¹ is —C₁₋₃ alkylene-.

44. The compound of claim 41, wherein L¹ is —C₁₋₃ alkylene-substituted with R¹⁰.

45. The compound of claim 41, wherein L¹ is selected from methylene, ethylene, propylene.

46. The compound of any one of claims 41-45, wherein the compound has formula: 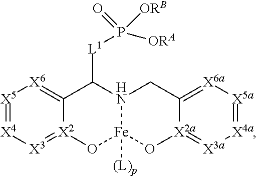
or a pharmaceutically acceptable salt thereof.

47. The compound of any one of claims 41-45, wherein the compound has formula: 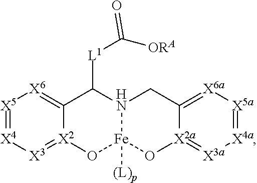
or a pharmaceutically acceptable salt thereof.

48. The compound of any one of claims 41-45, wherein the compound has formula: 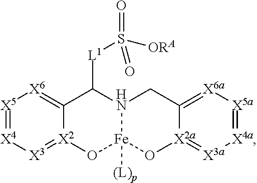
or a pharmaceutically acceptable salt thereof.

49. The compound of any one of claims 41-45, wherein the compound has formula: 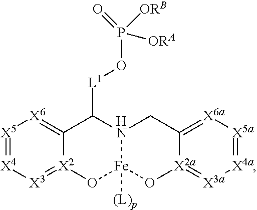
or a pharmaceutically acceptable salt thereof.

50. The compound of any one of claims 41-49, wherein the compound has formula: 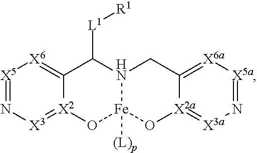
or a pharmaceutically acceptable salt thereof.

51. The compound of any one of claims 41-49, wherein the compound has formula: 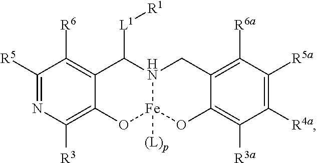
or a pharmaceutically acceptable salt thereof.

52. The compound of any one of claims 41-49, wherein the compound has formula: 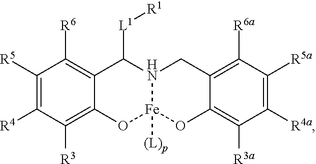
or a pharmaceutically acceptable salt thereof.

53. The compound of any one of claims 41-52, wherein at least one of R³, R^{3a}, R⁴, R^{4a}, R⁵, R^{5a}, R⁶, and R^{6a} is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸.

54. The compound of claim 53, wherein at least one of R³, R^{3a}, R⁴, R^{4a}, R⁵, R^{5a}, R⁶, and R^{6a} is selected from C₁₋₃ alkyl, —(C₁₋₃ alkyl)_{q}OH, —(C₁₋₃ alkyl)_{q}SO₃R^{A}, —(C₁₋₃ alkyl)_{q}CO₂R^{A}, —(C₁₋₃ alkyl)_{q}OPO₃R^{A}R^{B}, and —(C₁₋₃ alkyl)_{q}PO₃R^{A}R⁸.

55. The compound of claim 54, wherein at least one of R³, R^{3a}, R⁴, R^{4a}, R⁵, R^{5a}, R⁶, and R^{6a} is selected from C₁₋₃ alkyl, CH₂OH, C(O)OH, SO₃H, and CH₂OP(O)(OH)₂.

56. The compound of any one of claims 41-54, wherein q is 0.

57. The compound of any one of claims 41-54, wherein q is 1.

58. The compound of any one of claims 41-57, wherein each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.

59. A compound which is a Fe³⁺ complex of Formula (D-I): 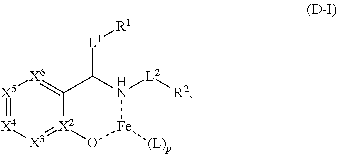
or a pharmaceutically acceptable salt thereof, wherein: [symbol]
indicates a coordinate bond;
each L is independently a Fe³⁺ ligand;
p is 0, 1 or 2;
L¹ is —C₁₋₃ alkylene-, or L¹ is absent;
L² is —C₁₋₃ alkylene-;
R¹ is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
R² is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
R³, R⁴, R⁵, and R⁶ and are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl), (C═O)NHSO₂R⁸;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN;
provided that the compound is not any one of the following compounds:

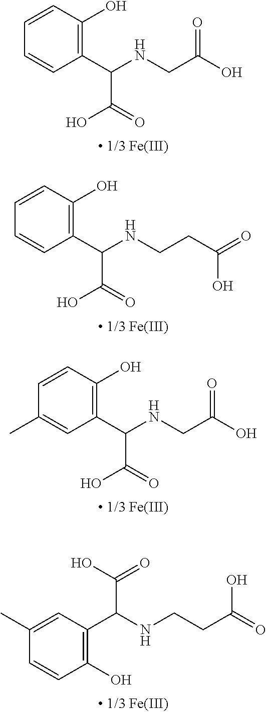 [SMILES: O=C(O)CNC(C(=O)O)c1ccccc1O ; O=C(O)CCNC(C(=O)O)c1ccccc1O ; Cc1ccc(O)c(C(NCC(=O)O)C(=O)O)c1 ; Cc1ccc(O)c(C(NCCC(=O)O)C(=O)O)c1]

60. The compound of claim 59, wherein L¹ is absent.

61. The compound of claim 59, wherein L¹ is —C₁₋₃ alkylene-.

62. The compound of claim 61, wherein L¹ is selected from methylene, ethylene, and propylene.

63. The compound of any one of claims 59-62, wherein L² is —C₁₋₃ alkylene-.

64. The compound of claim 63, wherein L² is selected from methylene, ethylene, and propylene.

65. The compound of any one of claims 59-64, wherein the compound has formula: 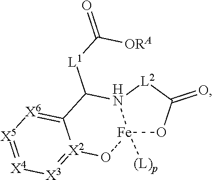
or a pharmaceutically acceptable salt thereof.

66. The compound of any one of claims 59-64, wherein the compound has formula: 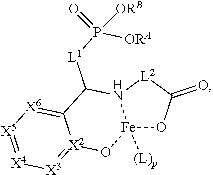
or a pharmaceutically acceptable salt thereof.

67. The compound of any one of claims 59-64, wherein the compound has formula: 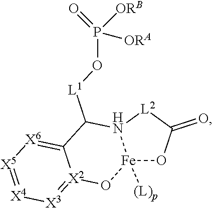
or a pharmaceutically acceptable salt thereof.

68. The compound of any one of claims 59-64, wherein the compound has formula: 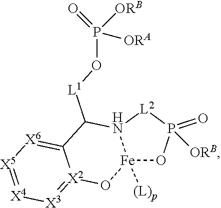
or a pharmaceutically acceptable salt thereof.

69. The compound of any one of claims 59-64, wherein the compound has formula: 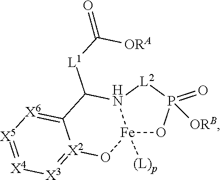
or a pharmaceutically acceptable salt thereof.

70. The compound of any one of claims 59-64, wherein the compound has formula: 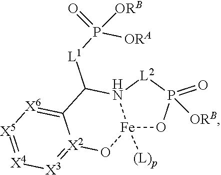
or a pharmaceutically acceptable salt thereof.

71. The compound of any one of claims 59-64, wherein the compound has formula: 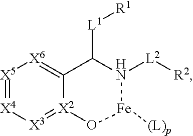
or a pharmaceutically acceptable salt thereof.

72. The compound of any one of claims 59-64, wherein the compound has formula: 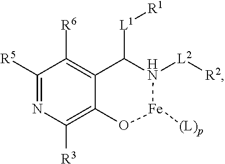
or a pharmaceutically acceptable salt thereof.

73. The compound of any one of claims 59-64, wherein the compound has formula: 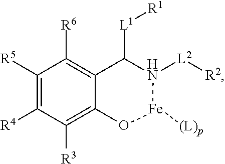
or a pharmaceutically acceptable salt thereof.

74. The compound of any one of claims 59-73, wherein at least one of R³, R⁴, R⁵, and R⁶ is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl), (C═O)NHSO₂R⁸.

75. The compound of claim 74, wherein at least one of R³, R^{3a}, R⁴, R^{4a}, R⁵, R^{5a}, R⁶, and R^{6a} is selected from C₁₋₃ alkyl, —(C₁₋₃ alkyl)_{q}OH, —(C₁₋₃ alkyl)_{q}SO₃R^{A}, —(C₁₋₃ alkyl)_{q}CO₂R^{A}, —(C₁₋₃ alkyl)_{q}OPO₃R^{A}R^{B}, and —(C₁₋₃ alkyl)_{q}PO₃R^{A}R⁸.

76. The compound of claim 75, wherein at least one of R³, R^{3a}, R⁴, R^{4a}, R⁵, R^{5a}, R⁶, and R^{6a} is selected from C₁₋₃ alkyl, CH₂OH, C(O)OH, SO₃H, and CH₂OP(O)(OH)₂.

77. The compound of any one of claims 59-75, wherein q is 0.

78. The compound of any one of claims 59-75, wherein q is 1.

79. The compound of any one of claims 59-78, wherein each L is independently selected from H₂O, NH₃, Cl, Br, SO₄, HCO₃, CO₃, PO₄, nitrate, nitrite, citric acid, tartaric acid, ascorbic acid, malic acid, succinic acid, acetic acid, glucose, fructose, mannose, and galactose, or any combination thereof.

80. A pharmaceutical composition comprising a compound of any one of claims 1-79, or a pharmaceutically acceptable salt thereof, and a pharmaceutically acceptable carrier.

81. A method of treating iron deficiency anemia, the method comprising administering to the subject in need thereof a therapeutically effective amount of a compound of any one of claims 1-79, or a pharmaceutically acceptable salt thereof, or a pharmaceutical composition of claim 80.

82. A compound of Formula (A): 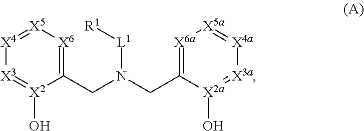
or a salt thereof, wherein:
L¹ is —C₁₋₃ alkylene-, optionally substituted with R¹⁰;
each R¹⁰ is independently —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl), (C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, or —(C₁₋₃ alkyl)_{q}(C═O)NHSO₂R⁸;
R¹ is selected from CO₂R^{A}, P(O)(OR^{A})(OR^{B}), and (C═O)NR^{A}R¹⁰;
each q is independently 0 or 1;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
X^{2a} is selected from N and C;
X^{3a} is selected from N and CR^{3a}, X^{4a} is selected from N and CR^{4a}, X^{5a} is selected from N and CR^{5a};
X^{6a} is selected from N and CR^{6a};
provided that no more than two of X^{2a}, X^{3a}, X^{4a}, X^{5a}, and X^{6a} are N;
R³, R^{3a}, R⁴, R^{4a}, R⁵, R^{5a}, R⁶, and R^{6a} are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰;
each R⁸ is selected from C_{3.9} alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C_{1.3} haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN,
provided that:
(i) if L¹R¹ is CH₂C(═O)OR^{A} or CH(CH₃)C(═O)OR^{A}, then:
X³ is different from X^{3a}; or
X⁴ is different from X^{4a}; or
X⁵ is different from X^{5a}, or
X⁶ is different from X^{6a}; or
X² is N; or
X^{2a} is N; and
(ii) the compound of Formula (A) is not any one of the following compounds:

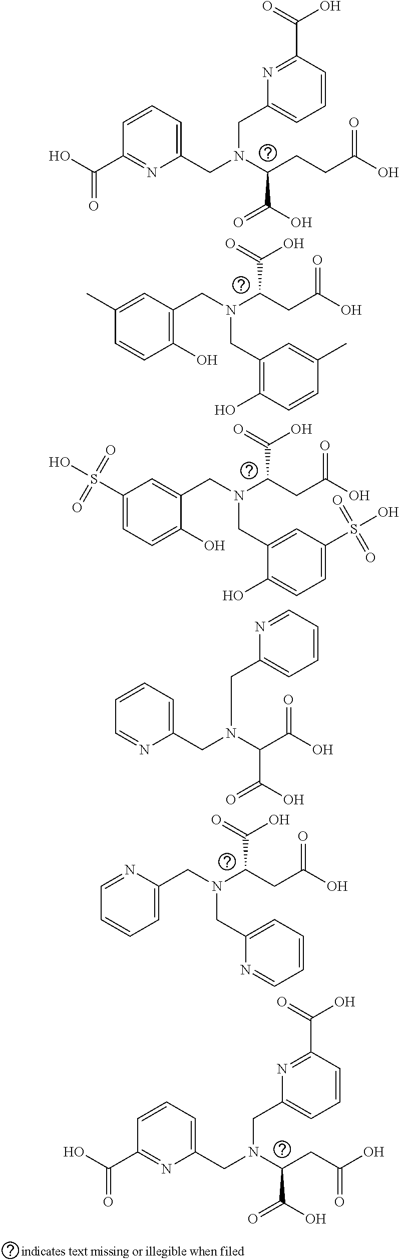

83. The compound of claim 82, having formula:

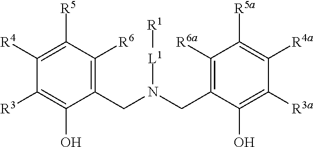

84. The compound of claim 82, having formula:

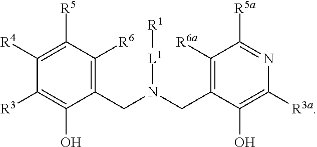

85. The compound of claim 82, having formula:

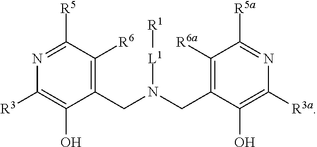

86. The compound of any one of claims 82-85, wherein L¹ is —C₁₋₃ alkylene-.

87. The compound of any one of claims 82-85, wherein L¹ is —C₁₋₃ alkylene-substituted with R¹⁰.

88. The compound of any one of claims 82-85, wherein L¹ is selected from methylene and ethylene.

89. The compound of any one of claims 82-85, having formula:

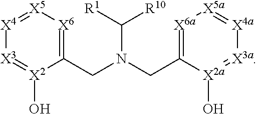

90. The compound of any one of claims 82-89, wherein R¹ is P(O)(OR^{A})(OR^{B}).

91. The compound of any one of claims 82-89, wherein R¹ is CO₂R^{A}.

92. The compound of any one of claims 82-89, wherein R¹ is (C═O)NR^{A}R¹⁰.

93. The compound of any one of claims 82-92, wherein R¹⁰ is selected from —(C₁₋₃ alkyl)_{q}CO₂R^{A}, —(C₁₋₃ alkyl)_{q}OP(R⁸)O₂R^{B}, —(C₁₋₃ alkyl), (C═O)NHSO₂R⁸, and —(C₁₋₃ alkyl)_{q}PO₃R^{A}R⁸.

94. The compound of any one of claims 82-92, wherein R¹⁰ is —(C₁₋₃ alkyl)_{q}CO₂R^{A}.

95. The compound of any one of claims 82-92, wherein R¹⁰ is —(C₁₋₃ alkyl)_{q}PO₃R^{A}R⁸.

96. The compound of any one of claims 82-95, wherein q is 0.

97. The compound of any one of claims 82-95, wherein q is 1.

98. The compound of claim 82, wherein the compound of formula A is selected from any one of the following compounds: 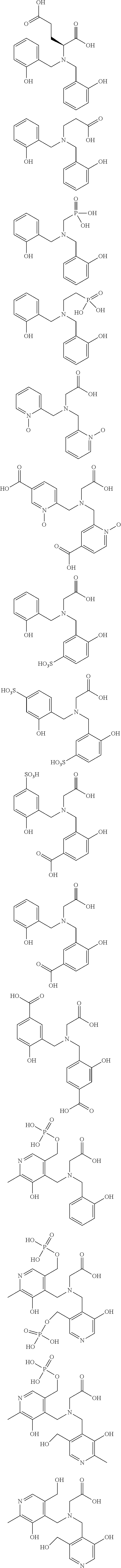
or a salt thereof.

99. A compound of Formula (B): 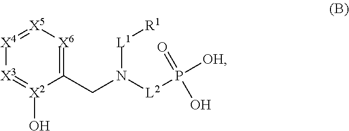
or a salt thereof, wherein:
L¹ is —C₁₋₃ alkylene-, optionally substituted with R¹⁰;
L² is —C₁₋₃ alkylene-, optionally substituted with R¹⁰;
each R¹⁰ is independently —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl), (C═O)NR^{A}R^{B}, —(C₁₋₃ alkyl)_{q}OP(R⁸)O₂R^{B}, —(C₁₋₃ alkyl)_{q}OPO: R^{A}R^{B}, —(C₁₋₃ alkyl)_{q}PO₃R^{A}R⁸, or —(C₁₋₃ alkyl)_{q}(C═O)NHSO₂R⁸;
R¹ is selected from CO₂R^{A}, P(O)(OR^{A})(OR^{B}), and (C═O)NR^{A}R¹⁰;
each q is independently 0 or 1;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
R³, R⁴, R⁵, and R⁶ and are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, and R¹⁰;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN,
provided that:
(i) if R¹ is P(OX(OH)(OH), then R⁵ is not C₁₋₃ alkyl or halo; and
(ii) the compound of Formula (A) is not any one of the following compounds:

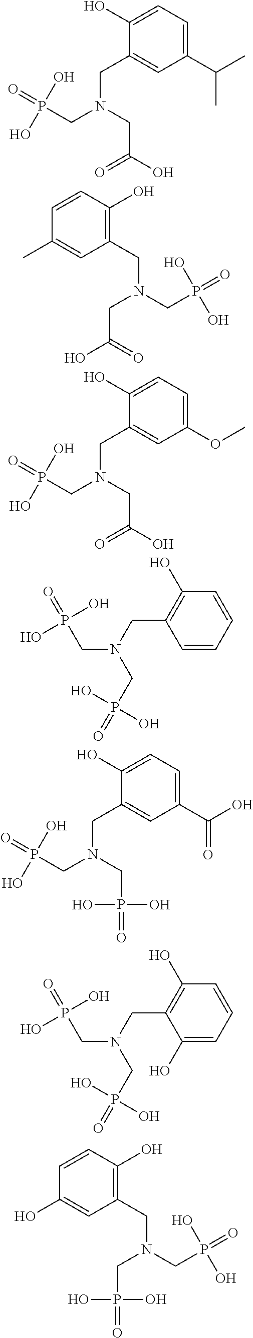

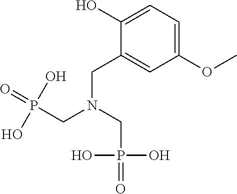 [SMILES: COc1ccc(O)c(CN(CP(=O)(O)O)CP(=O)(O)O)c1]

100. The compound of claim 99, wherein L² is —C₁₋₃ alkylene-.

101. The compound of claim 99, wherein L¹ is —C₁₋₃ alkylene-substituted with R¹⁰.

102. The compound of claim 99, wherein L¹ is selected from methylene and ethylene.

103. The compound of any one of claims 99-102, wherein L¹ is —C₁₋₃ alkylene-.

104. The compound of any one of claims 99-102, wherein L¹ is —C₁₋₃ alkylene-substituted with R¹⁰.

105. The compound of any one of claims 99-102, wherein L¹ is ethylene.

106. The compound of any one of claims 99-102, wherein the compound has formula:

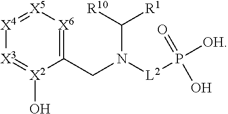

107. The compound of any one of claims 99-105, wherein the compound has formula: 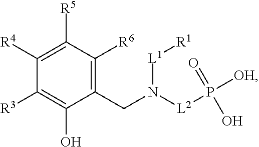
wherein R⁵ is selected from C₁₋₃ alkoxy, C₁₋₃ haloalkyl, C₁₋₃ haloalkoxy, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸.

108. The compound of any one of claims 99-105, wherein the compound has formula:

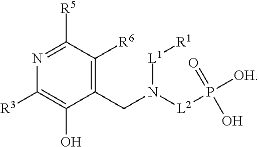

109. The compound of any one of claims 99-108, wherein R¹⁰ is selected from —(C₁₋₃ alkyl)_{q}CO₂R^{A}, —(C₁₋₃ alkyl)_{q}OP(R⁸)O₂R^{B}, —(C₁₋₃ alkyl), (C═O)NHSO₂R⁸, and —(C₁₋₃ alkyl)_{q}PO₃R^{A}R⁸.

110. The compound of claim 109, wherein R¹⁰ is —(C₁₋₃ alkyl)_{q}CO₂R^{A}.

111. The compound of claim 109, wherein R¹⁰ is —(C₁₋₃ alkyl)_{q}PO₃R^{A}R⁸.

112. The compound of any one of claims 99-111, wherein q is 0.

113. The compound of any one of claims 99-111, wherein q is 1.

114. The compound of claim 99, wherein the compound is selected from any one of the following compounds: 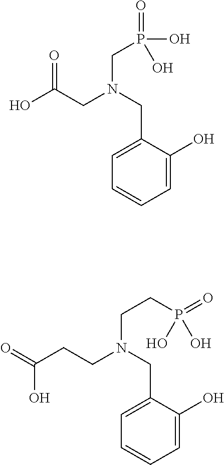 [SMILES: O=C(O)CN(Cc1ccccc1O)CP(=O)(O)O ; O=C(O)CCN(CCP(=O)(O)O)Cc1ccccc1O] 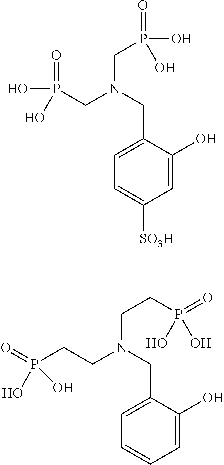 [SMILES: O=P(O)(O)CN(Cc1ccc(S(=O)(=O)O)cc1O)CP(=O)(O)O ; O=P(O)(O)CCN(CCP(=O)(O)O)Cc1ccccc1O]
or a salt thereof.

115. A compound of Formula (C): 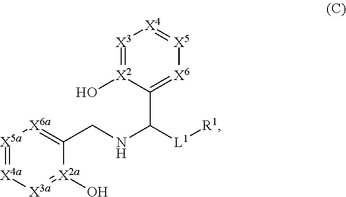
or a salt thereof, wherein:
L¹ is —C₁₋₃ alkylene-, or L¹ is absent;
R¹ is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X⁵, and X⁶ are N;
X^{2a} is selected from N and C;
X^{3a} is selected from N and CR^{3a};
X^{4a} is selected from N and CR^{4a};
X^{5a} is selected from N and CR^{5a};
X^{6a} is selected from N and CR^{6a};
provided that no more than two of X^{2a}, X^{3a}, X^{4a}, X^{5a}, and X^{6a} are N;
R³, R^{3a}, R⁴, R^{4a}, R⁵, R^{5a}, R⁶, and R^{6a} are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸;
each q is independently 0 or 1;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN.

116. The compound of claim 115, wherein L¹ is absent.

117. The compound of claim 115, wherein L¹ is —C₁₋₃ alkylene-.

118. The compound of claim 115, wherein L¹ is —C₁₋₃ alkylene-substituted with R¹⁰.

119. The compound of claim 115, wherein L¹ is selected from methylene, ethylene, propylene.

120. The compound of any one of claims 115-119, wherein the compound has formula: 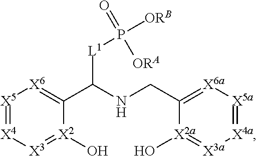
or a pharmaceutically acceptable salt thereof.

121. The compound of any one of claims 115-119, wherein the compound has formula: 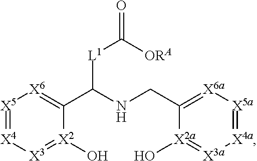
or a pharmaceutically acceptable salt thereof.

122. The compound of any one of claims 115-119, wherein the compound has formula: 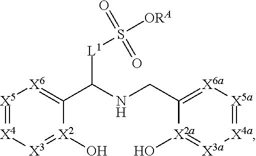
or a pharmaceutically acceptable salt thereof.

123. The compound of any one of claims 115-119, wherein the compound has formula: 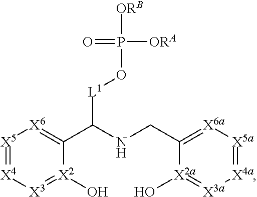
or a pharmaceutically acceptable salt thereof.

124. The compound of any one of claims 115-119, wherein the compound has formula: 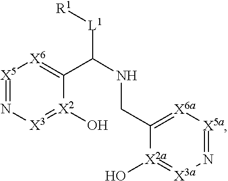
or a pharmaceutically acceptable salt thereof.

125. The compound of any one of claims 115-119, wherein the compound has formula: 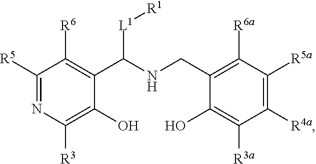
or a pharmaceutically acceptable salt thereof.

126. The compound of any one of claims 115-119, wherein the compound has formula: 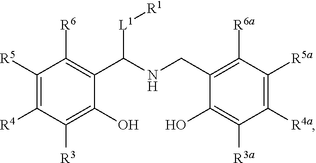
or a pharmaceutically acceptable salt thereof.

127. The compound of any one of claims 115-126, wherein at least one of R³, R^{3a}, R⁴, R^{4a}, R⁵, R^{5a}, R⁶, and R^{6a} is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl) NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸.

128. The compound of claim 127 wherein at least one of R³, R^{3a}, R⁴, R^{4a}, R⁵, R^{5a}, R⁶, and R^{6a} is selected from C₁₋₃ alkyl, —(C₁₋₃ alkyl)_{q}OH, —(C₁₋₃ alkyl)_{q}SO₃R^{A}, —(C₁₋₃ alkyl)_{q}CO₂R^{A}, —(C₁₋₃ alkyl)_{q}OPO₃R^{A}R^{B}, and —(C₁₋₃ alkyl)_{q}PO₃R^{A}R⁸.

129. The compound of claim 128, wherein at least one of R³, R^{3a}, R⁴, R^{4a}, R⁵, R^{5a}, R⁶, and R^{6a} is selected from C₁₋₃ alkyl, CH₂OH, C(O)OH, SO₃H, and CH₂OP(O)(OH)₂.

130. The compound of any one of claims 115-128, wherein q is 0.

131. The compound of any one of claims 115-128, wherein q is 1.

132. The compound of claim 115, wherein the compound of Formula (C) is selected from any one of the following compounds: 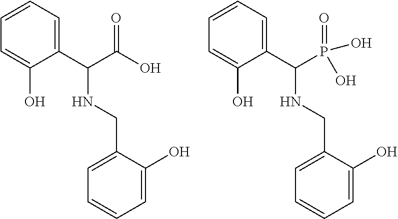 [SMILES: O=C(O)C(NCc1ccccc1O)c1ccccc1O ; O=P(O)(O)C(NCc1ccccc1O)c1ccccc1O]
or a salt thereof.

133. A compound of Formula (D): 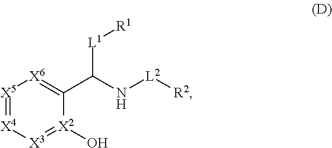
or a salt thereof, wherein:
L¹ is —Cd alkylene-, or L¹ is absent;
L² is —C₁₋₃ alkylene-;
R¹ is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
R² is selected from CO₂R^{A}, P(O)(OR^{A})(OR⁸), (C═O)NR^{A}R¹⁰, SO₃R^{A}, SO₂R⁸, NHSO₂R⁸, NR^{A}R^{B}, OP(R⁸)O₂R^{B}, OPO₃R^{A}R^{B}, and (C═O)NHSO₂R⁸;
provided that at least one of R¹ and R² is P(O)(OR^{A})(OR⁸);
each R^{A} and R^{B} are independently H or C₁₋₃ alkyl;
X² is selected from N and C;
X³ is selected from N and CR³;
X⁴ is selected from N and CR⁴;
X⁵ is selected from N and CR⁵;
X⁶ is selected from N and CR⁶;
provided that no more than two of X², X³, X⁴, X³, and X⁶ are N;
R³, R⁴, R⁵, and R⁶ and are each independently selected from H, C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl)_{q}(C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸;
each R⁸ is selected from C₃₋₉ alkyl and —(C₁₋₃ alkyl)_{q}C₆₋₁₀ aryl, wherein said C₆₋₁₀ aryl is optionally substituted with 1, 2, or 3 substituents independently selected from R¹¹; and
each R¹¹ is independently selected from C₁₋₆ alkyl, C₁₋₆ alkoxy, C₁₋₃ haloalkyl, C₁₋₃ haloalkoxy, halo, OH, CN, and C₆₋₁₀ aryl, which is optionally substituted with 1, 2, or 3 substituents independently selected from C₁₋₆ alkyl, C₁₋₆ alkoxy, C₁₋₃ haloalkyl, C₁₋₃ haloalkoxy, halo, OH, and CN;
provided that the compound of Formula (D) is not:

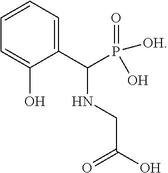 [SMILES: O=C(O)CNC(c1ccccc1O)P(=O)(O)O]

134. The compound of claim 133, wherein L¹ is absent.

135. The compound of claim 133, wherein L¹ is —C₁₋₃ alkylene-.

136. The compound of claim 135, wherein L¹ is selected from methylene, ethylene, and propylene.

137. The compound of any one of claims 133-136, wherein L² is —C₁₋₃ alkylene-.

138. The compound of claim 137, wherein L² is selected from methylene, ethylene, and propylene.

139. The compound of any one of claims 133-138, wherein the compound has formula: 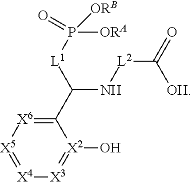
or a pharmaceutically acceptable salt thereof.

140. The compound of any one of claims 133-138, wherein the compound has formula: 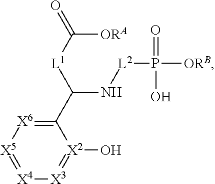
or a pharmaceutically acceptable salt thereof.

141. The compound of any one of claims 133-138, wherein the compound has formula: 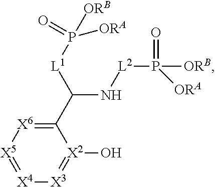
or a pharmaceutically acceptable salt thereof.

142. The compound of any one of claims 133-141, wherein the compound has formula: 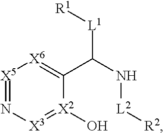
or a pharmaceutically acceptable salt thereof.

143. The compound of any one of claims 133-141, wherein the compound has formula: 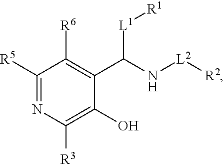
or a pharmaceutically acceptable salt thereof.

144. The compound of any one of claims 133-141, wherein the compound has formula: 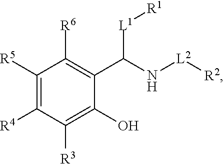
or a pharmaceutically acceptable salt thereof.

145. The compound of any one of claims 133-144, wherein at least one of R³, R⁴, R⁵, and R⁶ is selected from C₁₋₃ alkyl, C₁₋₃ alkoxy, C₁₋₃ haloalkyl, C₁₋₃ haloalkoxy, halo, —(C₁₋₃ alkyl)_{q}OH, —(C₁₋₃ alkyl)_{q}CN, —(C₁₋₃ alkyl)_{q}SO₃R^{A}, —(C₁₋₃ alkyl)_{q}SO₂R⁸, —(C₁₋₃ alkyl)_{q}NHSO₂R⁸, —(C₁₋₃ alkyl)_{q}CO₂R^{A}, —(C₁₋₃ alkyl)_{q}NR^{A}R^{B}, —(C₁₋₃ alkyl), (C═O)NR^{A}R^{B}, —(C₁₋₃ alkyl)_{q}OP(R⁸)O₂R^{B}, —(C₁₋₃ alkyl)_{q}OPO₃R^{A}R^{B}, —(C₁₋₃ alkyl)_{q}PO₃R^{A}R⁸, and —(C₁₋₃ alkyl)_{q}(C═O)NHSO₂R⁸.

146. The compound of claim 145, wherein at least one of R³, R^{3a}, R⁴, R^{4a}, R⁵, R^{5a}, R⁶, and R^{6a} is selected from C₁₋₃ alkyl, —(C₁₋₃ alkyl)_{q}OH, —(C₁₋₃ alkyl)_{q}SO₃R^{A}, —(C₁₋₃ alkyl)_{q}CO₂R^{A}, —(C₁₋₃ alkyl)_{q}OPO₃R^{A}R^{B}, and —(C₁₋₃ alkyl)_{q}PO₃R^{A}R⁸.

147. The compound of claim 146, wherein at least one of R³, R^{3a}, R⁴, R^{4a}, R⁵, R^{5a}, R⁶, and R^{6a} is selected from C₁₋₃ alkyl, CH₂OH, C(O)OH, SO₃H, and CH₂OP(O)(OH)₂.

148. The compound of any one of claims 133-146, wherein q is 0.

149. The compound of any one of claims 133-146, wherein q is 1.

150. The compound of claim 133, wherein the compound is selected from any one of the following compounds: 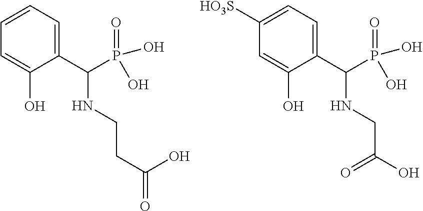 [SMILES: O=C(O)CCNC(c1ccccc1O)P(=O)(O)O ; O=C(O)CNC(c1ccc(S(=O)(=O)O)cc1O)P(=O)(O)O]
or a salt thereof.
